(19) United States
(12) Patent Application Publication
(10) Pub. No.: US 2022/0304984 A1
(43) Pub. Date: Sep. 29, 2022

(54) AMINO ACID TRANSPORT INHIBITORS AND THE USES THEREOF

(71) Applicant: VANDERBILT UNIVERSITY, Nasville, TN (US)

(72) Inventors: H. Charles Manning, Nashville, TN (US); Michael Schulte, Nashville, TN (US)

(21) Appl. No.: 17/618,829

(22) PCT Filed: Jun. 12, 2020
(86) PCT No.: PCT/US2020/037549

§ 371 Date: Dec. 13, 2021

Related U.S. Application Data
(60) Provisional application No. 62/860,677, filed on Jun. 12, 2019.

(51) Int. Cl. A61K 31/42; C07D 209/42; C07D 307/85; C07D 333/70; C07D 235/24; C07D 263/58; C07D 277/68; C07D 471/04; C07D 491/048; C07D 495/04; C07D 498/04; C07D 513/04; C07D 487/04; C07D 473/00; C07D 207/34; C07D 333/40; C07D 307/68; C07D 231/20; C07D 275/03; C07D 261/18; C07D 233/90; C07D 277/56; C07D 263/40; C07D 249/12; C07D 285/08; C07D 271/07; C07D 285/13; C07D 271/113; A61K 45/06; A61K 31/404; A61K 31/343; A61K 31/381; A61K 31/4184; A61K 31/423; A61K 31/428; A61K 31/437; A61K 31/4355; A61K 31/4365; A61K 31/519; A61K 31/52; A61K 31/401; A61K 31/341; A61K 31/415; A61K 31/425; A61K 31/4164; A61K 31/426; A61K 31/421; A61K 31/4196; A61K 31/433; A61K 31/4245

(52) U.S. Cl. CPC A61K 31/42; C07D 209/42; C07D 307/85; C07D 333/70; C07D 235/24; C07D 263/58; C07D 277/68; C07D 471/04; C07D 491/048; C07D 495/04; C07D 498/04; C07D 513/04; C07D 487/04; C07D 473/00; C07D 207/34; C07D 333/40; C07D 307/68; C07D 231/20; C07D 275/03; C07D 261/18; C07D 233/90; C07D 277/56; C07D 263/40; C07D 249/12; C07D 285/08; C07D 271/07; C07D 285/13; C07D 271/113; A61K 45/06; A61K 31/404; A61K 31/343; A61K 31/381; A61K 31/4184; A61K 31/423; A61K 31/428; A61K 31/437; A61K 31/4355; A61K 31/4365; A61K 31/519; A61K 31/52; A61K 31/401; A61K 31/341; A61K 31/415; A61K 31/425; A61K 31/4164; A61K 31/426; A61K 31/421; A61K 31/4196; A61K 31/433; A61K 31/4245

(57) ABSTRACT

These compounds are glutamine transporter inhibitors, e.g., alanine, serine, cysteine-preferring transporter 2 (ASCT2) inhibitors. Glutamine transporter inhibitors are useful to treat a variety of diseases, disorders, or conditions including cancer.

### FIELD OF THE INVENTION

The present disclosure provides compounds as amino acid transport inhibitors, e.g., glutamine transport inhibitors, e.g., alanine, serine, cysteine transporter 2 (ASCT2) inhibitors. Amino acid transport inhibitors are useful to treat a variety of diseases, disorders, or conditions including cancer.

### BACKGROUND OF THE INVENTION

Glutamine and other amino acids are involved in multiple aspects of cancer metabolism (Hensley et al., J Clin. Invest. 123:3678-3684 (2013)). For example, glutamine is the most abundant amino acid in the blood and muscle and is utilized for energy generation. Glutamine is also a precursor for the biomass required for rapid cancer cell proliferation (Windmueller and Spaeth, J. Biol. Chem. 249:5070-5079 (1974)). In addition to providing a carbon source, glutamine metabolism also acts as a source of nitrogen for the synthesis of nucleic acids and other amino acids. Glutamine also participates in the regulation of cellular redox homeostasis through a variety of mechanisms (Altman et al., Nat. Rev. Cancer 16:773 (2016)). Cancer cells are thus dependent on glutamine and cannot survive in the absence of exogenous glutamine. Choi and Park, Biomol Ther 26(1):19-28 (2018).

Several membrane transport proteins in humans ensure glutamine homeostasis by coordinating glutamine's absorption, reabsorption, and delivery to tissues. Amino acid transporters include ASCT2, BOAT1, SNAT1, SNAT2, SNAT3, SNAT5, SNAT7, LAT1, and LAT2. See, e.g., Pochini et al., Frontiers in Chemistry 2 (Article 61):1-22 (2014); Bhutia and Ganapathy, Biochimica et Biophysica Acta 1863:2531-2539 (2016).

ASCT2 is a cell surface solute-carrying transporter that mediates uptake of neutral amino acids including glutamine (Kanai and Hediger, Pflugers Arch 447:469-479 (2004); Kekuda et al., J Biol Chem 271:18657-18661 (1996)). Blocking ASCT2 to prevent glutamine uptake has been shown to successfully prevent tumor cell proliferation in melanoma (Wang Q et al., Int J Cancer 135:1060-1071 (2014)), non-small cell lung cancer (Hassanein et al., Clin Cancer Res 19:560-570 (2013); Hassanein et al., Int J Cancer 137:1587-1597 (2015)), prostate cancer (Wang et al., J Pathol 236: 278-289 (2015)), acute myeloid leukemia (Willems et al., Blood 122: 3521-3532 (2013)), and triple-negative breast cancer (van Geldermalsen et al., Oncogene 35, 3201-3208 (2016)).

ASCT2 inhibitors are disclosed in WO 2018/107173, Schulte et al., Bioorg Med Chem Lett 25(1):113-116 (2015), Schulte et al., Bioorg Med Chem Lett 26(3):1044-1047 (2016), and Schulte et al., Nat Med 24(2):194-202 (2018). In light of the importance of glutamine in cancer cell biology, there exists a need in the art for new ASCT2 and other glutamine transporter inhibitors. See, e.g., Scalise et al., Front Cell Dev Biol 6:96 (2018).

### BRIEF SUMMARY OF THE INVENTION

In one aspect, the present disclosure provides compounds represented by any one of Formulae I-IV, below, and the pharmaceutically acceptable salts and solvates thereof, collectively referred to herein as “Compounds of the Disclosure.”

In another aspect, the present disclosure provides a pharmaceutical composition comprising a Compound of the Disclosure and one or more pharmaceutically acceptable carriers.

In another aspect, the present disclosure provides a method of inhibiting one or more amino acid, e.g., glutamine, transporters, e.g., ASCT2, BOAT1, SNAT1, SNAT2, SNAT3, SNAT5, SNAT7, LAT1, and/or LAT2, in a subject, comprising administering to the subject an effective amount of at least one Compound of the Disclosure.

In another aspect, the present disclosure provides methods for treating a disease, disorder, or condition in a subject, comprising administering a therapeutically effective amount of a Compound of the Disclosure to the subject.

In another aspect, the present disclosure provides methods for treating a disease, disorder, or condition in a subject, comprising administering a therapeutically effective amount of a Compound of the Disclosure in combination with one or more optional therapeutic agents to the subject.

In another aspect, the present disclosure provides methods for treating a disease, disorder, or condition responsive to inhibition of one or more amino acid, e.g., glutamine, transporters, e.g., ASCT2, BOAT1, SNAT1, SNAT2, SNAT3, SNAT5, SNAT7, LAT1, and/or LAT2, comprising administering a therapeutically effective amount of a Compound of the Disclosure to a subject.

In another aspect, the present disclosure provides the use of a Compound of the Disclosure to inhibit one or more amino acid, e.g., glutamine, transporters, e.g., ASCT2, BOAT1, SNAT1, SNAT2, SNAT3, SNAT5, SNAT7, LAT1, and/or LAT2.

In another aspect, the present disclosure provides a pharmaceutical composition for treating a disease, disorder, or condition in a subject, wherein the pharmaceutical composition comprises a therapeutically effective amount of a Compound of the Disclosure in a mixture with one or more pharmaceutically acceptable carriers.

In another aspect, the present disclosure provides Compounds of the Disclosure for use in treating cancer in a subject in need thereof.

In another aspect, the present disclosure provides a Compound of the Disclosure for use in the manufacture of a medicament for treating cancer in a mammal.

In another aspect, the present disclosure provides a therapeutic or prophylactic agent for cancer, which comprises a Compound of the Disclosure.

In another aspect, the present disclosure provides a kit comprising a Compound of the Disclosure.

In another aspect, the present disclosure provides a method of treating a subject having cancer, the method comprising administering a therapeutically effective amount of a Compound of the Disclosure to the subject if a mutation in any one or more of BRAF, KRAS, p53, and/or PI3KCA, is present in a biological sample of the subject.

In another aspect, the present disclosure provides a method of treating a subject having cancer, the method comprising administering a therapeutically effective amount of a Compound of the Disclosure to the subject if an overexpression of MYC is present in a biological sample of the subject.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### BRIEF DESCRIPTION OF THE DRAWINGS

FIG. 1 is a bar graph showing the glutamine uptake inhibition of Cpd. Nos. 2, 10, 45, and 47 at the concentrations indicated.

### DETAILED DESCRIPTION OF THE INVENTION

### I. Compounds of the Disclosure

In one embodiment, Compounds of the Disclosure are compounds of Formula I.

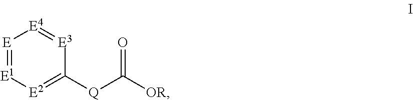

wherein:

R is selected from the group consisting of hydrogen, C₁-C₄ alkyl, and aralkyl;

E is selected from the group consisting of ═C(R⁵)—, ═C(R^{1a})—, and ═N—;

E¹ is selected from the group consisting of ═C(R⁵)—, ═C(R^{1a})—, and ═N—;

with the proviso that one of E or E¹ is ═C(R⁵)— and the other is ═C(R^{1a})— or ═N—;

E² is selected from the group consisting of ═C(R^{1b})— and ═N—;

E³ is selected from the group consisting of ═C(R^{1c})— and ═N—;

E⁴ is selected from the group consisting of ═C(R^{1d})— and ═N—;

R^{1a}, R^{1b}, R^{1c}, and R^{1d} are each independently selected from the group consisting of hydrogen, halogen, cyano, C₁-C₄ alkyl, C₁-C₄ haloalkyl, and C₁-C₄ alkoxy;

R⁵ is selected from the group consisting of optionally substituted aryl and optionally substituted heteroaryl;

Q is selected from the group consisting of:

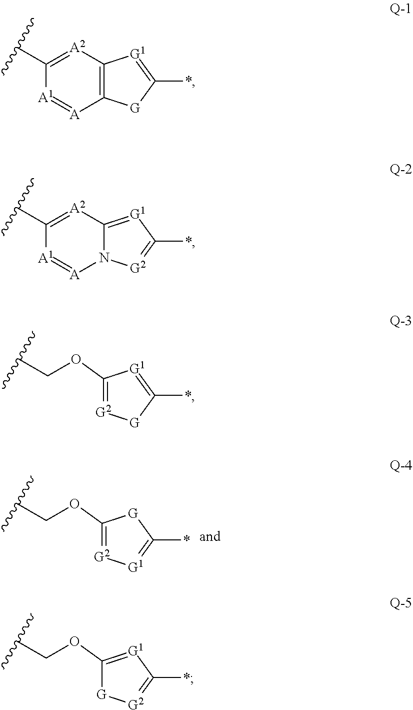

with the proviso that the bond designated with an “*” is attached to —C(═O)OR;

G is selected from the group consisting of —S—, —O—, and —N(R^{2a})—;

G¹ is selected from the group consisting of ═C(R^{3a})— and ═N—;

G² is selected from the group consisting of ═C(R^{3b})— and ═N—;

A is selected from the group consisting of ═C(R^{4a})— and ═N—;

A¹ is selected from the group consisting of ═C(R^{4b})— and ═N—;

A² is selected from the group consisting of ═C(R^{4c})— and ═N—;

R^{2a} is selected from the group consisting of hydrogen and C₁-C₄ alkyl;

R^{3a} and R^{3b} are independently selected from the group consisting of hydrogen, hydroxy, halo, C₁-C₄ alkyl, and C₁-C₄ haloalkyl; and

R^{4a}, R^{4b}, and R^{4c} are each independently selected from the group consisting of hydrogen, halogen, cyano, C₁-C₄ alkyl, C₁-C₄ haloalkyl, and C₁-C₄ alkoxy;

or a pharmaceutically acceptable salt or solvate thereof.

In another embodiment, the compound of Formula I is not:
5-([1,1′-biphenyl]-4-yl)-6-chloro-1H-pyrrolo[3,2-b]pyridine-2-carboxylic acid;
1-methyl-3-((4-(5-(trifluoromethyl)-1,2,4-oxadiazol-3-yl)benzyl)oxy)-1H-pyrazole-5-carboxylic acid; or
3-((4-(4-phenyl-1H-indol-1-yl)benzyl)oxy)isoxazole-5-carboxylic acid.

In another embodiment, Compounds of the Disclosure are compounds of Formula II:

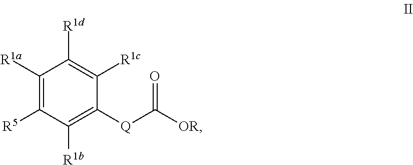

wherein R^{1a}, R^{1b}, R^{1c}, R^{1d}, R⁵, Q, and R are as defined in connection with Formula I, or a pharmaceutically acceptable salt or solvate thereof.

In another embodiment, Compounds of the Disclosure are compounds of Formula III:

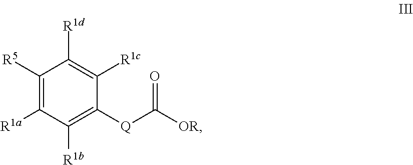

wherein R^{1a}, R^{1b}, R^{1c}, R^{1d}, R⁵, Q, and R are as defined in connection with Formula I, or a pharmaceutically acceptable salt or solvate thereof.

In another embodiment, Compounds of the Disclosure are compounds of any one of Formulae I-III, wherein R⁵ is optionally substituted heteroaryl, or a pharmaceutically acceptable salt or solvate thereof.

In another embodiment, Compounds of the Disclosure are compounds of any one of Formulae I-III, wherein R⁵ is optionally substituted aryl, or a pharmaceutically acceptable salt or solvate thereof.

In another embodiment, Compounds of the Disclosure are compounds of Formula IV:

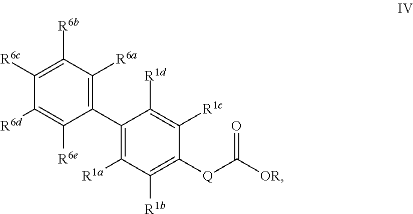

wherein R^{6a}, R^{6b}, R^{6c}, R^{6d}, and R^{6e} are each independently selected from the group consisting of hydrogen, halogen, cyano, C₁-C₄ alkyl, C₁-C₄ haloalkyl, and C₁-C₄ alkoxy; and R^{1a}, R^{1b}, R^{1c}, R^{1d}, Q, and R are as defined in connection with Formula I or a pharmaceutically acceptable salt or solvate thereof.

In another embodiment, Compounds of the Disclosure are compounds of Formula IV, wherein R^{1a}, R^{1b}, R^{1c}, R^{1d}, R^{6d}, and R^{6e} are each hydrogen, or a pharmaceutically acceptable salt or solvate thereof.

In another embodiment, Compounds of the Disclosure are compounds of any one of Formulae I-IV, wherein Q is Q-1, or a pharmaceutically acceptable salt or solvate thereof. In another embodiment Q-1 is selected from the group consisting of:

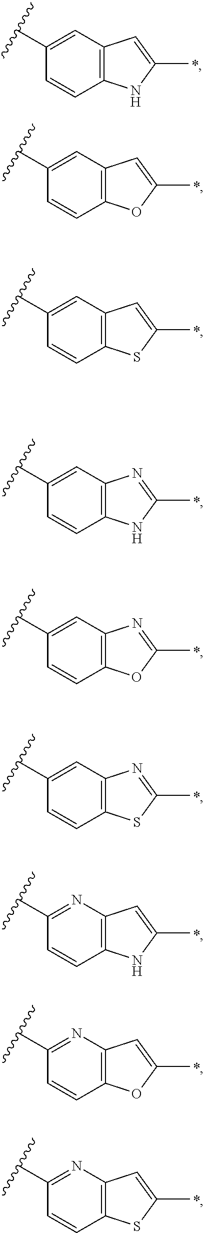

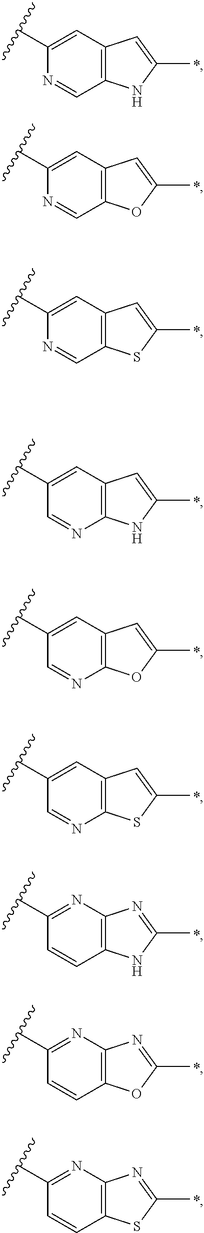

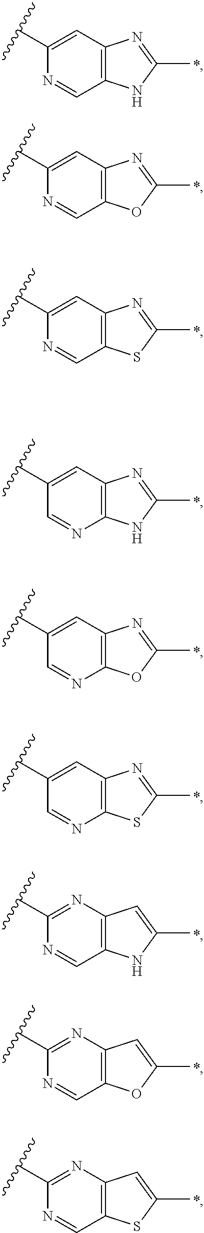

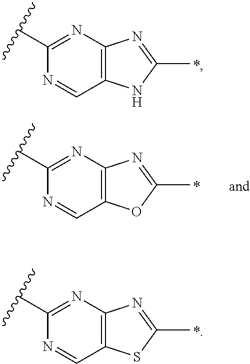 [SMILES: *c1ncc2[nH]c(C)nc2n1 ; *c1ncc2oc(C)nc2n1 ; *c1ncc2sc(C)nc2n1]

In another embodiment, Compounds of the Disclosure are compounds of any one of Formulae I-IV, wherein Q is Q-2, or a pharmaceutically acceptable salt or solvate thereof. In another embodiment, Q-2 is selected from the group consisting of

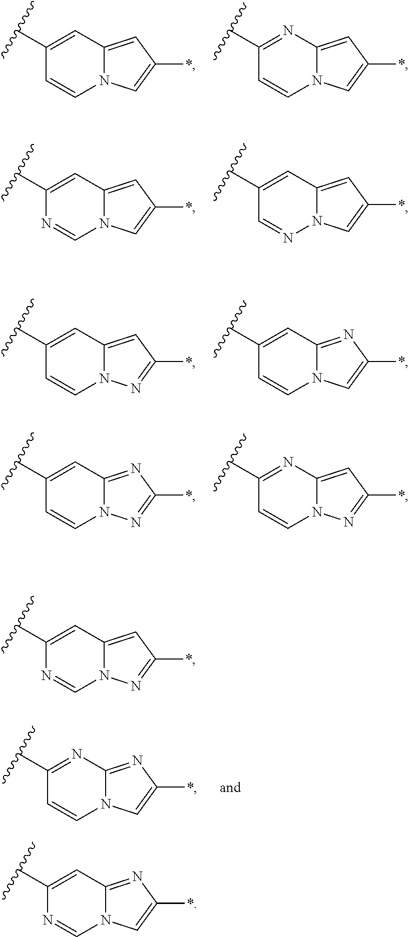

In another embodiment, Compounds of the Disclosure are compounds of any one of Formulae I-IV, wherein Q is Q-3, or a pharmaceutically acceptable salt or solvate thereof. In another embodiment, Q-3 is selected from the group consisting of:

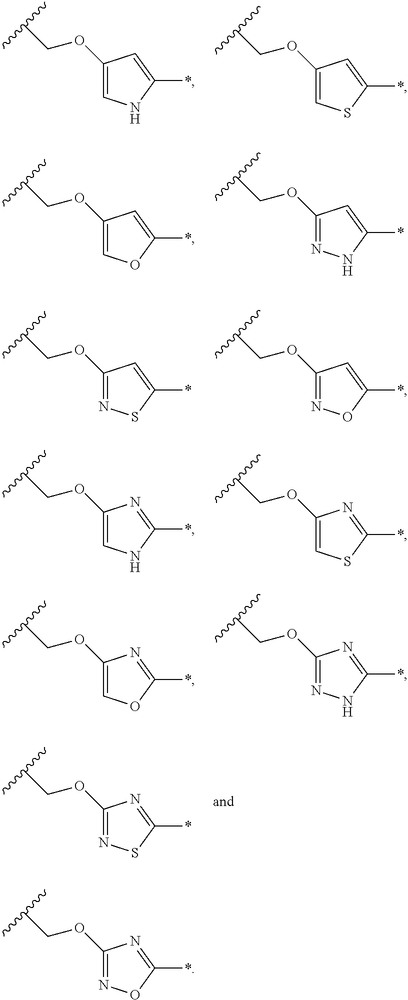

In another embodiment, Compounds of the Disclosure are compounds of any one of Formulae I-IV, wherein Q is Q-4, or a pharmaceutically acceptable salt or solvate thereof. In another embodiment, Q-4 is selected from the group consisting of:

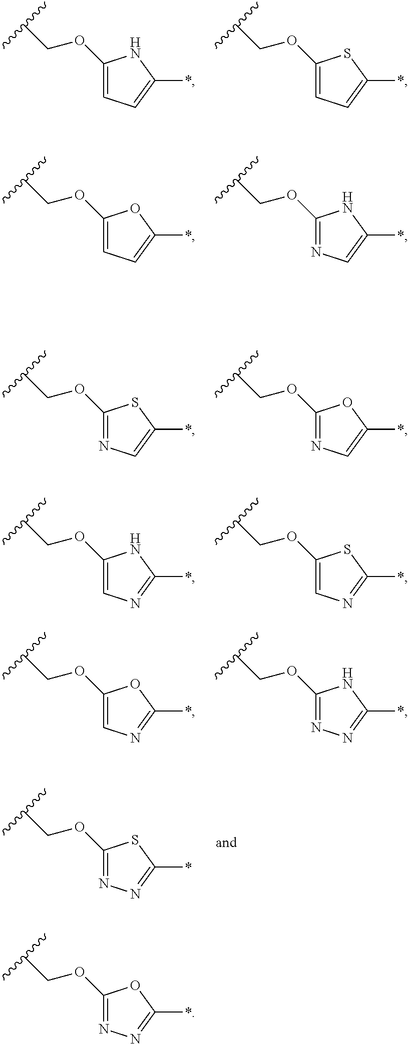

In another embodiment, Compounds of the Disclosure are compounds of any one of Formulae I-IV, wherein Q is Q-5, or a pharmaceutically acceptable salt or solvate thereof. In another embodiment, Q-5 is selected from the group consisting of:

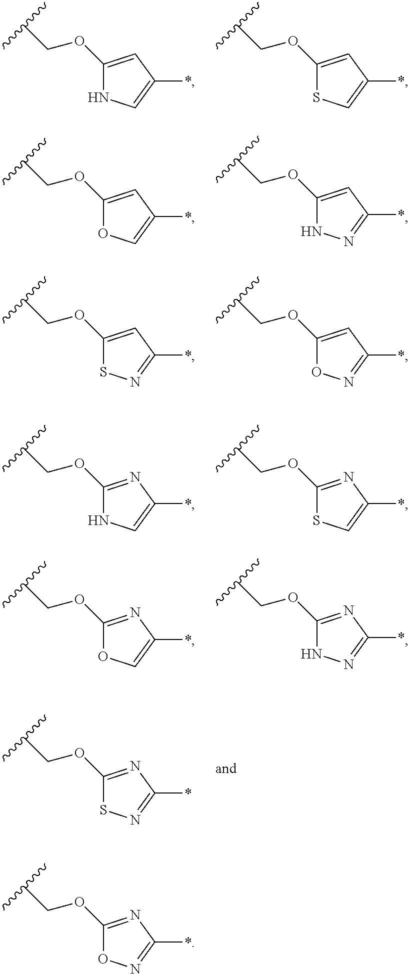

In another embodiment, Compounds of the Disclosure are compounds of any one of Formulae I-IV, wherein R is hydrogen, or a pharmaceutically acceptable salt or solvate thereof.

In another embodiment, Compounds of the Disclosure are compounds selected from any one or more of the compounds of Table 1, or a pharmaceutically acceptable salt or solvate thereof.

TABLE 1

| Cpd. | | |
|---|---|---|
| No. | Structure | Name |
| 1 | 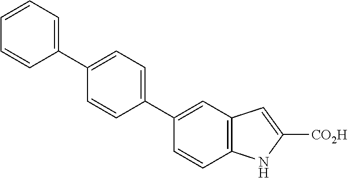 [SMILES: O=C(O)c1cc2cc(-c3ccc(-c4ccccc4)cc3)ccc2[nH]1] | 5-([1,1′-biphenyl]-4-yl)-1H-indole-2- carboxylic acid |
| 2 | 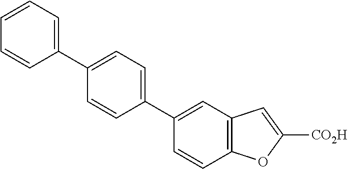 [SMILES: O=C(O)c1cc2cc(-c3ccc(-c4ccccc4)cc3)ccc2o1] | 5-([1,1′-biphenyl]-4-yl)benzofuran-2- carboxylic acid |
| 3 | 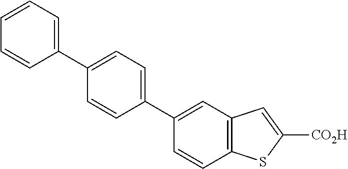 [SMILES: O=C(O)c1cc2cc(-c3ccc(-c4ccccc4)cc3)ccc2s1] | 5-([1,1′-biphenyl]-4-yl)benzo[b]thiophene-2- carboxylic acid |
| 4 | 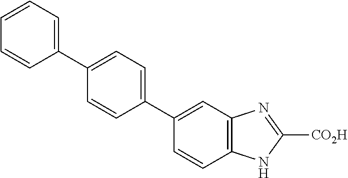 [SMILES: O=C(O)c1nc2cc(-c3ccc(-c4ccccc4)cc3)ccc2[nH]1] | 5-([1,1′-biphenyl]-4-yl)-1H- benzo[d]imidazole-2-carboxylic acid |
| 5 | 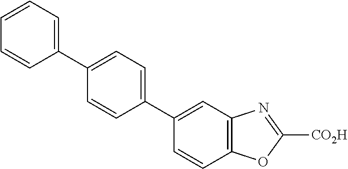 [SMILES: O=C(O)c1nc2cc(-c3ccc(-c4ccccc4)cc3)ccc2o1] | 5-([1,1′-biphenyl]-4-yl)benzo[d]oxazole-2- carboxylic acid |
| 6 | 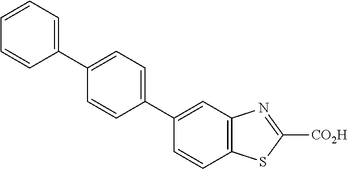 [SMILES: O=C(O)c1nc2cc(-c3ccc(-c4ccccc4)cc3)ccc2s1] | 5-([1,1′-biphenyl]-4-yl)benzo[d]thiazole-2- carboxylic acid |
| 7 | 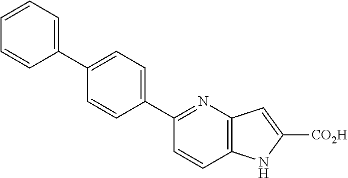 [SMILES: O=C(O)c1cc2nc(-c3ccc(-c4ccccc4)cc3)ccc2[nH]1] | 5-([1,1′-biphenyl]-4-yl)-1H-pyrrolo[3,2- b]pyridine-2-carboxylic acid |
| 8 | 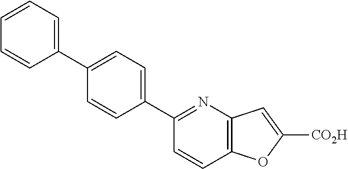 [SMILES: O=C(O)c1cc2nc(-c3ccc(-c4ccccc4)cc3)ccc2o1] | 5-([1,1′-biphenyl]-4-yl)furo[3,2-b]pyridine-2- carboxylic acid |
| 9 | 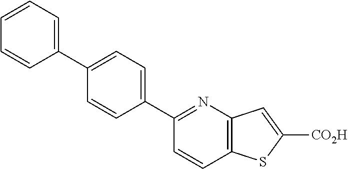 [SMILES: O=C(O)c1cc2nc(-c3ccc(-c4ccccc4)cc3)ccc2s1] | 5-([1,1′-biphenyl]-4-yl)thieno[3,2-b]pyridine- 2-carboxylic acid |
| 10 | 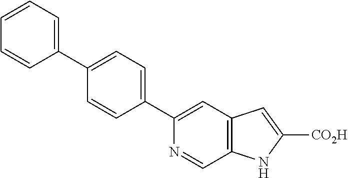 [SMILES: O=C(O)c1cc2cc(-c3ccc(-c4ccccc4)cc3)ncc2[nH]1] | 5-([1,1′-biphenyl]-4-yl)-1H-pyrrolo[2,3- c]pyridine-2-carboxylic acid |
| 11 | 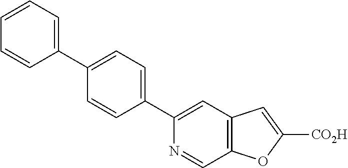 [SMILES: O=C(O)c1cc2cc(-c3ccc(-c4ccccc4)cc3)ncc2o1] | 5-([1,1′-biphenyl]-4-yl)furo[2,3-c]pyridine-2- carboxylic acid |
| 12 | 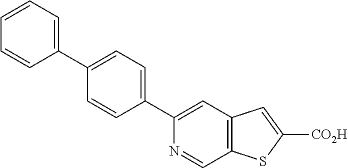 [SMILES: O=C(O)c1cc2cc(-c3ccc(-c4ccccc4)cc3)ncc2s1] | 5-([1,1′-biphenyl]-4-yl)thieno[2,3-c]pyridine- 2-carboxylic acid |
| 13 | 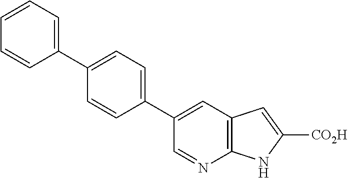 [SMILES: O=C(O)c1cc2cc(-c3ccc(-c4ccccc4)cc3)cnc2[nH]1] | 5-([1,1′-biphenyl]-4-yl)-1H-pyrrolo[2,3- b]pyridine-2-carboxylic acid |
| 14 | 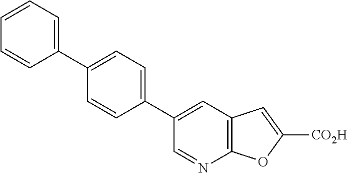 [SMILES: O=C(O)c1cc2cc(-c3ccc(-c4ccccc4)cc3)cnc2o1] | 5-([1,1′-biphenyl]-4-yl)furo[2,3-b]pyridine-2- carboxylic acid |
| 15 | 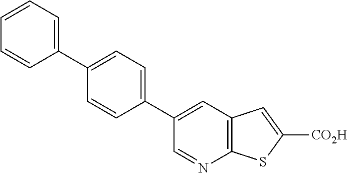 [SMILES: O=C(O)c1cc2cc(-c3ccc(-c4ccccc4)cc3)cnc2s1] | 5-([1,1′-biphenyl]-4-yl)thieno[2,3-b]pyridine- 2-carboxylic acid |
| 16 | 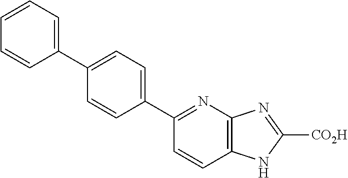 [SMILES: O=C(O)c1nc2nc(-c3ccc(-c4ccccc4)cc3)ccc2[nH]1] | 5-([1,1′-biphenyl]-4-yl)-1H-imidazo[4,5- b]pyridine-2-carboxylic acid |
| 17 | 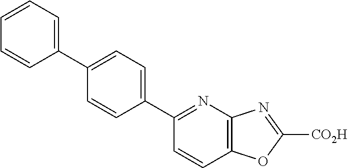 [SMILES: O=C(O)c1nc2nc(-c3ccc(-c4ccccc4)cc3)ccc2o1] | 5-([1,1′-biphenyl]-4-yl)oxazolo[4,5- b]pyridine-2-carboxylic acid |
| 18 | 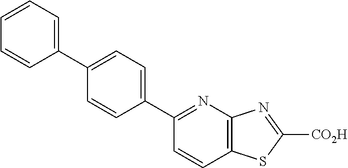 [SMILES: O=C(O)c1nc2nc(-c3ccc(-c4ccccc4)cc3)ccc2s1] | 5-([1,1′-biphenyl]-4-yl)thiazolo[4,5- b]pyridine-2-carboxylic acid |
| 19 | 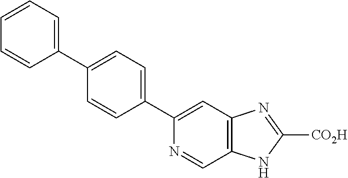 [SMILES: O=C(O)c1nc2cc(-c3ccc(-c4ccccc4)cc3)ncc2[nH]1] | 6-([1,1′-biphenyl]-4-yl)-3H-imidazo[4,5- c]pyridine-2-carboxylic acid |
| 20 | 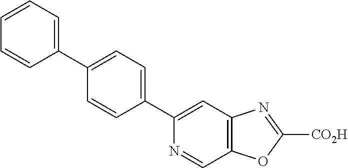 [SMILES: O=C(O)c1nc2cc(-c3ccc(-c4ccccc4)cc3)ncc2o1] | 6-([1,1′-biphenyl]-4-yl)oxazolo[5,4- c]pyridine-2-carboxylic acid |
| 21 | 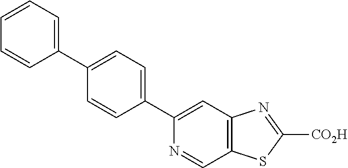 [SMILES: O=C(O)c1nc2cc(-c3ccc(-c4ccccc4)cc3)ncc2s1] | 6-([1,1′-biphenyl]-4-yl)thiazolo[5,4- c]pyridine-2-carboxylic acid |
| 22 | 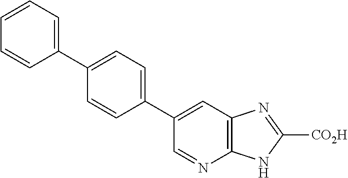 [SMILES: O=C(O)c1nc2cc(-c3ccc(-c4ccccc4)cc3)cnc2[nH]1] | 6-([1,1′-biphenyl]-4-yl)-3H-imidazo[4,5- b]pyridine-2-carboxylic acid |
| 23 | 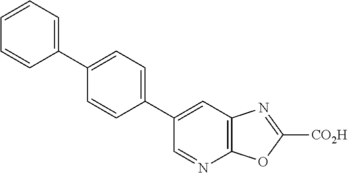 [SMILES: O=C(O)c1nc2cc(-c3ccc(-c4ccccc4)cc3)cnc2o1] | 6-([1,1′-biphenyl]-4-yl)oxazolo[5,4- b]pyridine-2-carboxylic acid |
| 24 | 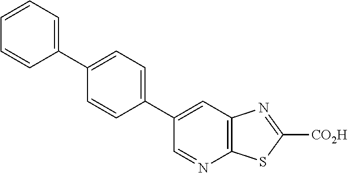 [SMILES: O=C(O)c1nc2cc(-c3ccc(-c4ccccc4)cc3)cnc2s1] | 6-([1,1′-biphenyl]-4-yl)thiazolo[5,4- b]pyridine-2-carboxylic acid |
| 25 | 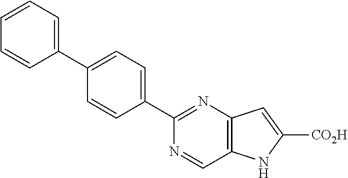 [SMILES: O=C(O)c1cc2nc(-c3ccc(-c4ccccc4)cc3)ncc2[nH]1] | 2-([1,1′-biphenyl]-4-yl)-5H-pyrrolo[3,2- d]pyrimidine-6-carboxylic acid |
| 26 | 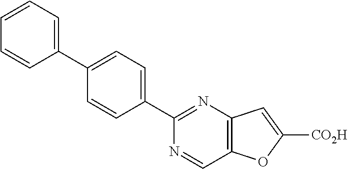 [SMILES: O=C(O)c1cc2nc(-c3ccc(-c4ccccc4)cc3)ncc2o1] | 2-([1,1′-biphenyl]-4-yl)furo[3,2-d]pyrimidine- 6-carboxylic acid |
| 27 | 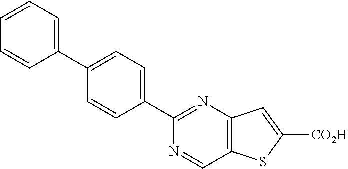 [SMILES: O=C(O)c1cc2nc(-c3ccc(-c4ccccc4)cc3)ncc2s1] | 2-([1,1′-biphenyl]-4-yl)thieno[3,2- d]pyrimidine-6-carboxylic acid |
| 28 | 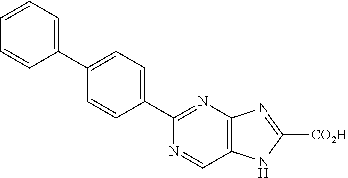 [SMILES: O=C(O)c1nc2nc(-c3ccc(-c4ccccc4)cc3)ncc2[nH]1] | 2-([1,1′-biphenyl]-4-yl)-7H-purine-8- carboxylic acid |
| 29 | 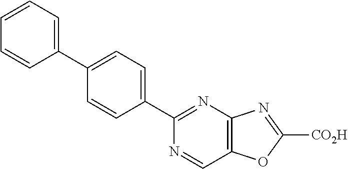 [SMILES: O=C(O)c1nc2nc(-c3ccc(-c4ccccc4)cc3)ncc2o1] | 5-([1,1′-biphenyl]-4-yl)oxazolo[4,5- d]pyrimidine-2-carboxylic acid |
| 30 | 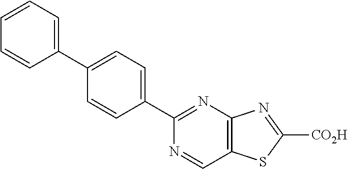 [SMILES: O=C(O)c1nc2nc(-c3ccc(-c4ccccc4)cc3)ncc2s1] | 5-([1,1′-biphenyl]-4-yl)thiazolo[4,5- d]pyrimidine-2-carboxylic acid |
| 31 | 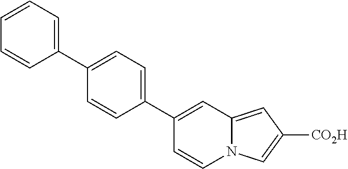 [SMILES: O=C(O)c1cc2cc(-c3ccc(-c4ccccc4)cc3)ccn2c1] | 7-([1,1′-biphenyl]-4-yl)indolizine-2- carboxylic acid |
| 32 | 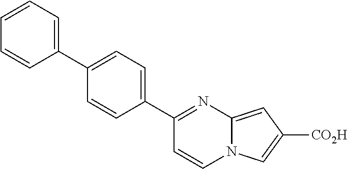 [SMILES: O=C(O)c1cc2nc(-c3ccc(-c4ccccc4)cc3)ccn2c1] | 2-([1,1′-biphenyl]-4-yl)pyrrolo[1,2- a]pyrimidine-7-carboxylic acid |
| 33 | 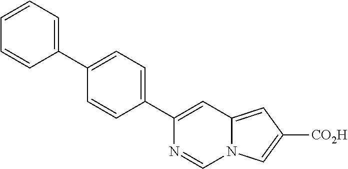 [SMILES: O=C(O)c1cc2cc(-c3ccc(-c4ccccc4)cc3)ncn2c1] | 3-([1,1′-biphenyl]-4-yl)pyrrolo[1,2- c]pyrimidine-6-carboxylic acid |
| 34 | 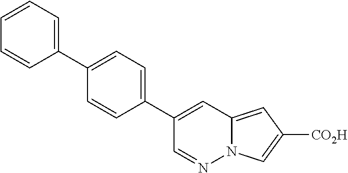 [SMILES: O=C(O)c1cc2cc(-c3ccc(-c4ccccc4)cc3)cnn2c1] | 3-([1,1′-biphenyl]-4-yl)pyrrolo[1,2- b]pyridazine-6-carboxylic acid |
| 35 | 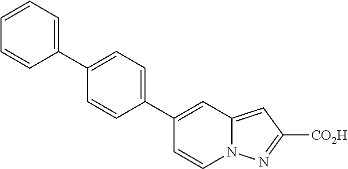 [SMILES: O=C(O)c1cc2cc(-c3ccc(-c4ccccc4)cc3)ccn2n1] | 5-([1,1′-biphenyl]-4-yl)pyrazolo[1,5- a]pyridine-2-carboxylic acid |
| 36 | 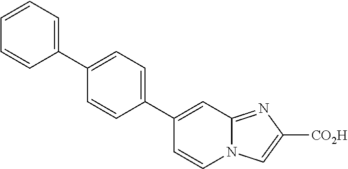 [SMILES: O=C(O)c1cn2ccc(-c3ccc(-c4ccccc4)cc3)cc2n1] | 7-([1,1′-biphenyl]-4-yl)imidazo[1,2- a]pyridine-2-carboxylic acid |
| 37 | 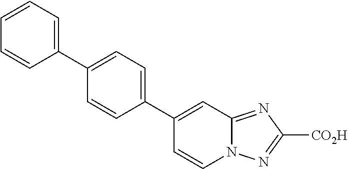 [SMILES: O=C(O)c1nc2cc(-c3ccc(-c4ccccc4)cc3)ccn2n1] | 7-([1,1′-biphenyl]-4-yl)-[1,2,4]triazolo[1,5- a]pyridine-2-carboxylic acid |
| 38 | 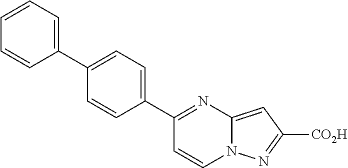 [SMILES: O=C(O)c1cc2nc(-c3ccc(-c4ccccc4)cc3)ccn2n1] | 5-([1,1′-biphenyl]-4-yl)pyrazolo[1,5- a]pyrimidine-2-carboxylic acid |
| 39 | 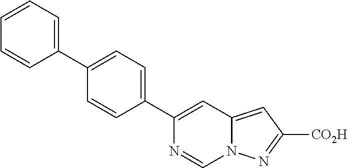 [SMILES: O=C(O)c1cc2cc(-c3ccc(-c4ccccc4)cc3)ncn2n1] | 5-([1,1′-biphenyl]-4-yl)pyrazolo[1,5- c]pyrimidine-2-carboxylic acid |
| 40 | 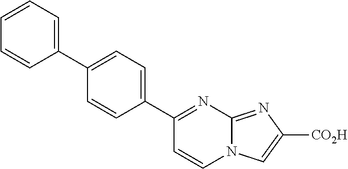 [SMILES: O=C(O)c1cn2ccc(-c3ccc(-c4ccccc4)cc3)nc2n1] | 7-([1,1′-biphenyl]-4-yl)imidazo[1,2- a]pyrimidine-2-carboxylic acid |
| 41 | 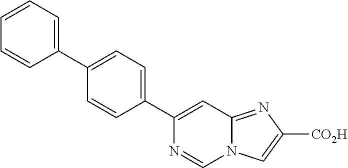 [SMILES: O=C(O)c1cn2cnc(-c3ccc(-c4ccccc4)cc3)cc2n1] | 7-([1,1′-biphenyl]-4-yl)imidazo[1,2- c]pyrimidine-2-carboxylic acid |
| 42 | 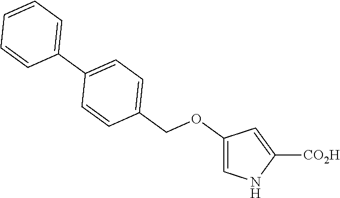 [SMILES: O=C(O)c1cc(OCc2ccc(-c3ccccc3)cc2)c[nH]1] | 4-([1,1′-biphenyl]-4-ylmethoxy)-1H-pyrrole-2- carboxylic acid |
| 43 | 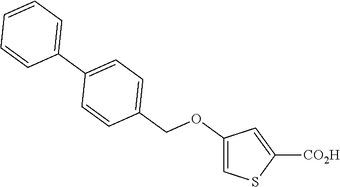 [SMILES: O=C(O)c1cc(OCc2ccc(-c3ccccc3)cc2)cs1] | 4-([1,1′-biphenyl]-4-ylmethoxy)thiophene-2- carboxylic acid |
| 44 | 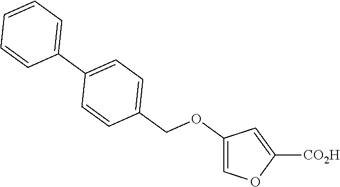 [SMILES: O=C(O)c1cc(OCc2ccc(-c3ccccc3)cc2)co1] | 4-([1,1′-biphenyl]-4-ylmethoxy)furan-2- carboxylic acid |
| 45 | 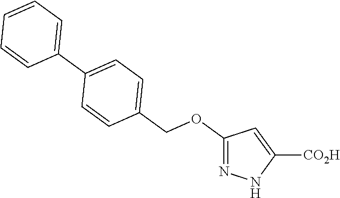 [SMILES: O=C(O)c1cc(OCc2ccc(-c3ccccc3)cc2)n[nH]1] | 3-([1,1′-biphenyl]-4-ylmethoxy)-1H-pyrazole- 5-carboxylic acid |
| 46 | 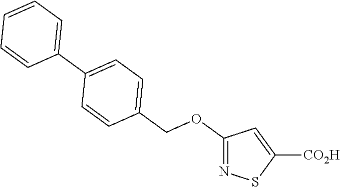 [SMILES: O=C(O)c1cc(OCc2ccc(-c3ccccc3)cc2)ns1] | 3-([1,1′-biphenyl]-4-ylmethoxy)isothiazole-5- carboxylic acid |
| 47 | 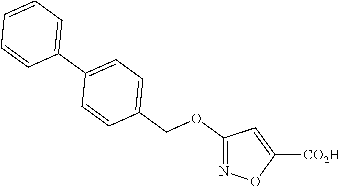 [SMILES: O=C(O)c1cc(OCc2ccc(-c3ccccc3)cc2)no1] | 3-([1,1′-biphenyl]-4-ylmethoxy)isoxazole-5- carboxylic acid |
| 48 | 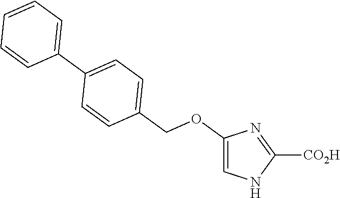 [SMILES: O=C(O)c1nc(OCc2ccc(-c3ccccc3)cc2)c[nH]1] | 4-([1,1′-biphenyl]-4-ylmethoxy)-1H- imidazole-2-carboxylic acid |
| 49 | 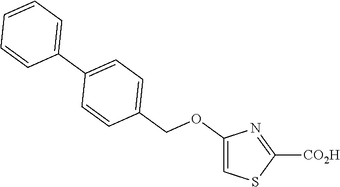 [SMILES: O=C(O)c1nc(OCc2ccc(-c3ccccc3)cc2)cs1] | 4-([1,1′-biphenyl]-4-ylmethoxy)thiazole-2- carboxylic acid |
| 50 | 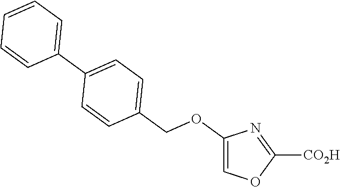 [SMILES: O=C(O)c1nc(OCc2ccc(-c3ccccc3)cc2)co1] | 4-([1,1′-biphenyl]-4-ylmethoxy)oxazole-2- carboxylic acid |
| 51 | 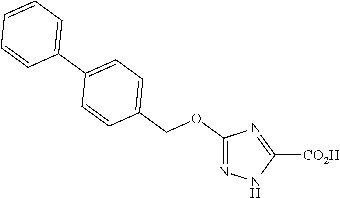 [SMILES: O=C(O)c1nc(OCc2ccc(-c3ccccc3)cc2)n[nH]1] | 3-([1,1′-biphenyl]-4-ylmethoxy)-1H-1,2,4- triazole-5-carboxylic acid |
| 52 | 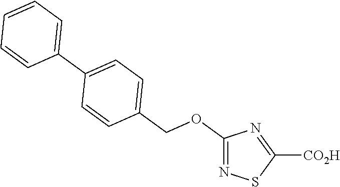 [SMILES: O=C(O)c1nc(OCc2ccc(-c3ccccc3)cc2)ns1] | 3-([1,1′-biphenyl]-4-ylmethoxy)-1,2,4- thiadiazole-5-carboxylic acid |
| 53 | 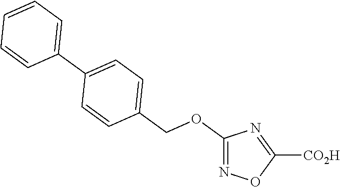 [SMILES: O=C(O)c1nc(OCc2ccc(-c3ccccc3)cc2)no1] | 3-([1,1′-biphenyl]-4-ylmethoxy)-1,2,4- oxadiazole-5-carboxylic acid |
| 54 | 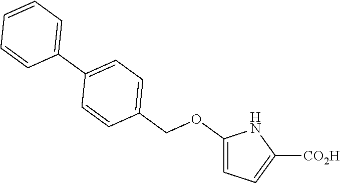 [SMILES: O=C(O)c1ccc(OCc2ccc(-c3ccccc3)cc2)[nH]1] | 5-([1,1′-biphenyl]-4-ylmethoxy)-1H-pyrrole-2- carboxylic acid |
| 55 | 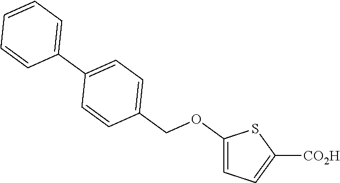 [SMILES: O=C(O)c1ccc(OCc2ccc(-c3ccccc3)cc2)s1] | 5-([1,1′-biphenyl]-4-ylmethoxy)thiophene-2- carboxylic acid |
| 56 | 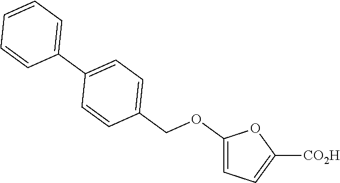 [SMILES: O=C(O)c1ccc(OCc2ccc(-c3ccccc3)cc2)o1] | 5-([1,1′-biphenyl]-4-ylmethoxy)furan-2- carboxylic acid |
| 57 | 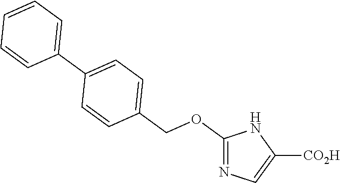 [SMILES: O=C(O)c1cnc(OCc2ccc(-c3ccccc3)cc2)[nH]1] | 2-([1,1′-biphenyl]-4-ylmethoxy)-1H- imidazole-5-carboxylic acid |
| 58 | 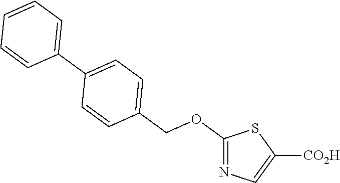 [SMILES: O=C(O)c1cnc(OCc2ccc(-c3ccccc3)cc2)s1] | 2-([1,1′-biphenyl]-4-ylmethoxy)thiazole-5- carboxylic acid |
| 59 | 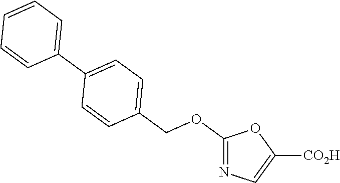 [SMILES: O=C(O)c1cnc(OCc2ccc(-c3ccccc3)cc2)o1] | 2-([1,1′-biphenyl]-4-ylmethoxy)oxazole-5- carboxylic acid |
| 60 | 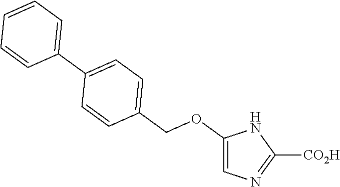 [SMILES: O=C(O)c1ncc(OCc2ccc(-c3ccccc3)cc2)[nH]1] | 5-([1,1′-biphenyl]-4-ylmethoxy)-1H- imidazole-2-carboxylic acid |
| 61 | 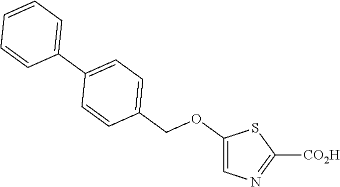 [SMILES: O=C(O)c1ncc(OCc2ccc(-c3ccccc3)cc2)s1] | 5-([1,1′-biphenyl]-4-ylmethoxy)thiazole-2- carboxylic acid |
| 62 | 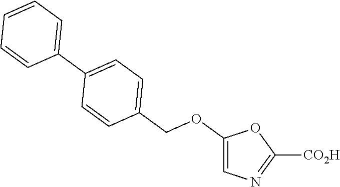 [SMILES: O=C(O)c1ncc(OCc2ccc(-c3ccccc3)cc2)o1] | 5-([1,1′-biphenyl]-4-ylmethoxy)oxazole-2- carboxylic acid |
| 63 | 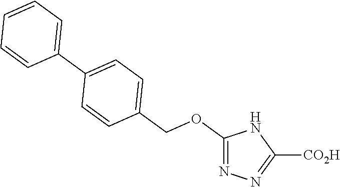 [SMILES: O=C(O)c1nnc(OCc2ccc(-c3ccccc3)cc2)[nH]1] | 5-([1,1′-biphenyl]-4-ylmethoxy)-4H-1,2,4- triazole-3-carboxylic acid |
| 64 | 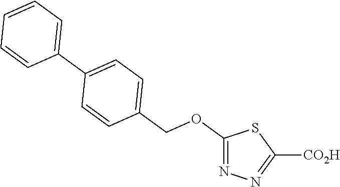 [SMILES: O=C(O)c1nnc(OCc2ccc(-c3ccccc3)cc2)s1] | 5-([1,1′-biphenyl]-4-ylmethoxy)-1,3,4- thiadiazole-2-carboxylic acid |
| 65 | 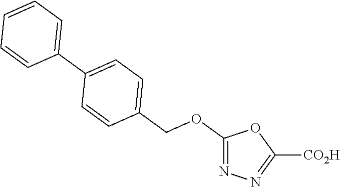 [SMILES: O=C(O)c1nnc(OCc2ccc(-c3ccccc3)cc2)o1] | 5-([1,1′-biphenyl]-4-ylmethoxy)-1,3,4- oxadiazole-2-carboxylic acid |
| 66 | 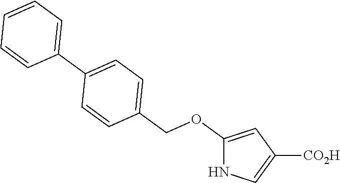 [SMILES: O=C(O)c1c[nH]c(OCc2ccc(-c3ccccc3)cc2)c1] | 5-([1,1′-biphenyl]-4-ylmethoxy)-1H-pyrrole-3- carboxylic acid |
| 67 | 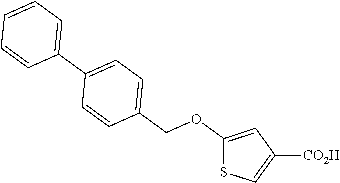 [SMILES: O=C(O)c1csc(OCc2ccc(-c3ccccc3)cc2)c1] | 5-([1,1′-biphenyl]-4-ylmethoxy)thiophene- 3-carboxylic acid |
| 68 | 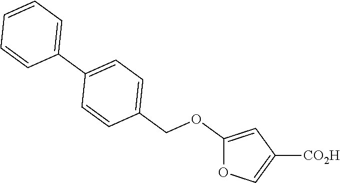 [SMILES: O=C(O)c1coc(OCc2ccc(-c3ccccc3)cc2)c1] | 5-([1,1′-biphenyl]-4-ylmethoxy)furan- 3-carboxylic acid |
| 69 | 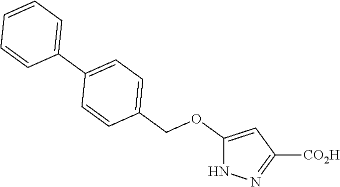 [SMILES: O=C(O)c1cc(OCc2ccc(-c3ccccc3)cc2)[nH]n1] | 5-([1,1′-biphenyl]-4-ylmethoxy)-1H- pyrazole-3-carboxylic acid |
| 70 | 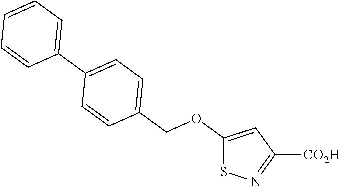 [SMILES: O=C(O)c1cc(OCc2ccc(-c3ccccc3)cc2)sn1] | 5-([1,1′-biphenyl]-4-ylmethoxy)isothiazole- 3-carboxylic acid |
| 71 | 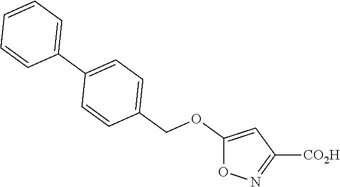 [SMILES: O=C(O)c1cc(OCc2ccc(-c3ccccc3)cc2)on1] | 5-([1,1′-biphenyl]-4-ylmethoxy)isoxazole- 3-carboxylic acid |
| 72 | 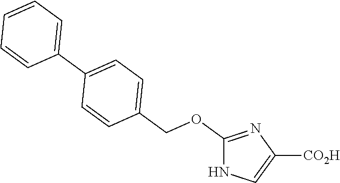 [SMILES: O=C(O)c1c[nH]c(OCc2ccc(-c3ccccc3)cc2)n1] | 2-([1,1′-biphenyl]-4-ylmethoxy)-1H- imidazole-4-carboxylic acid |
| 73 | 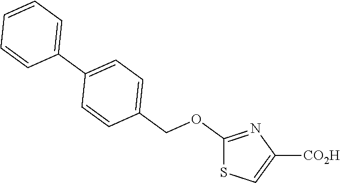 [SMILES: O=C(O)c1csc(OCc2ccc(-c3ccccc3)cc2)n1] | 2-([1,1′-biphenyl]-4-ylmethoxy)thiazole- 4-carboxylic acid |
| 74 | 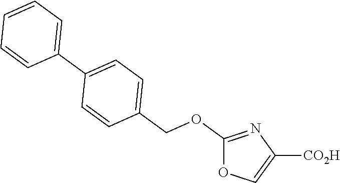 [SMILES: O=C(O)c1coc(OCc2ccc(-c3ccccc3)cc2)n1] | 2-([1,1′-biphenyl]-4-ylmethoxy)oxazole- 4-carboxylic acid |
| 75 | 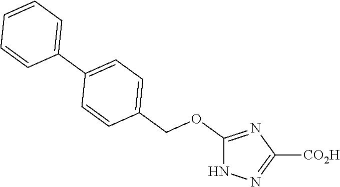 [SMILES: O=C(O)c1n[nH]c(OCc2ccc(-c3ccccc3)cc2)n1] | 5-([1,1′-biphenyl]-4-ylmethoxy)-1H-1,2,4- triazole-3-carboxylic acid |
| 76 | 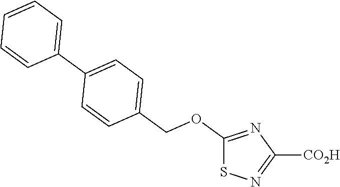 [SMILES: O=C(O)c1nsc(OCc2ccc(-c3ccccc3)cc2)n1] | 5-([1,1′-biphenyl]-4-ylmethoxy)-1,2,4- thiadiazole-3-carboxylic acid |
| 77 | 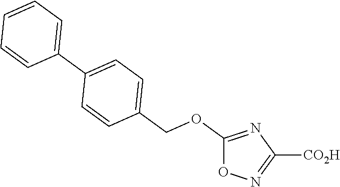 [SMILES: O=C(O)c1noc(OCc2ccc(-c3ccccc3)cc2)n1] | 5-([1,1′-biphenyl]-4-ylmethoxy)-1,2,4- oxadiazole-3-carboxylic acid |

Salts, hydrates, and solvates of the Compounds of the Disclosure can also be used in the methods disclosed herein. The present disclosure further includes all possible stereoisomers and geometric isomers of Compounds of the Disclosure to include both racemic compounds and optically active isomers. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure, including pharmaceutically acceptable salts. As used herein, the pharmaceutical “pharmaceutically acceptable salt” refers to salts or zwitterionic forms of Compounds of the Disclosure. Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the use of solvates of a Compound of the Disclosure. Solvates typically do not significantly alter the physiological activity or toxicity of a compound, and as such may function as pharmacological equivalents. The term “solvate” as used herein is a combination, physical association and/or solvation of a Compound of the Disclosure with a solvent molecule such as, e.g., a disolvate, monosolvate or hemisolvate, where the ratio of solvent molecule a Compound of the Disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. A Compound of the Disclosure can be present as solvated forms with a pharmaceutically acceptable solvent, such as water, methanol, ethanol, and the like, and it is intended that the disclosure includes both solvated and unsolvated forms of a Compound of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate involves dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure and Kits

Compounds of the Disclosure inhibit ASCT2-, BOAT1-, SNAT1-, SNAT2-, SNAT3-, SNAT5-, SNAT7-, LAT1-, and/or LAT2-mediated amino acid, e.g., glutamine, transport and thus are useful in the treatment of a variety of diseases, disorders, and conditions. In particular, Compounds of the Disclosure are useful in methods of treating diseases, disorders, and conditions wherein inhibition of ASCT2-, BOAT1-, SNAT1-, SNAT2-, SNAT3-, SNAT5-, SNAT7-, LAT1-, and/or LAT2-mediated glutamine transport provides a benefit. Diseases, disorders, and conditions treatable by the methods of the present disclosure include, but are not limited to, cancer and other proliferative disorders. Compounds of the Disclosure typically bind to ASCT2, BOAT1, SNAT1, SNAT2, SNAT3, SNAT5, SNAT7, LAT1, and/or LAT2 with an inhibition constant (Kᵢ) of less than 500 μM, e.g., less than 300 μM, less than 200 μM, less than 100 μM, less than 50 μM, less than 25 μM, less than 5 μM, or less than about 1 μM.

In one embodiment, the disclosure provides therapeutic methods, uses, and compositions relating to the treatment of cancer. These methods, uses, and compositions comprise administering a therapeutically effective amount of a Compound of the Disclosure to a subject in need thereof.

In another embodiment, a Compound of the Disclosure is administered to a subject having cancer as a single chemotherapeutic agent.

In another embodiment, a Compound of the Disclosure is administered to a subject having cancer in combination with one or more optional therapeutic agents. A Compound of the Disclosure and optional therapeutic agent(s) can be administered in combination under one or more of the following conditions: at different periodicities, at different durations, at different concentrations, by different administration routes, etc. In some embodiments, a Compound of the Disclosure is administered to the patient according to an intermittent dosing schedule.

In some embodiments, a Compound of the Disclosure is administered prior to the optional therapeutic agent(s), e.g., 0.5, 1, 2, 3, 4, 5, 10, 12, or 18 hours, 1, 2, 3, 4, 5, or 6 days, or 1, 2, 3, or 4 weeks prior to the administration of the immune checkpoint inhibitor.

In some embodiments, a Compound of the Disclosure is administered after the optional therapeutic agent(s), e.g., 0.5, 1, 2, 3, 4, 5, 10, 12, or 18 hours, 1, 2, 3, 4, 5, or 6 days, or 1, 2, 3, or 4 weeks after the administration of the immune checkpoint inhibitor.

In some embodiments, a Compound of the Disclosure and the optional therapeutic agent(s) are administered concurrently but on different schedules, e.g., a Compound of the Disclosure is administered daily while the optional therapeutic agent(s) is administered once a week, once every two weeks, once every three weeks, or once every four weeks. In other embodiments, a Compound of the Disclosure is administered once a day while the optional therapeutic agent(s) is administered once a week, once every two weeks, once every three weeks, or once every four weeks.

The therapeutic methods provided herein comprise administering a Compound of the Disclosure to a cancer patient in an amount which is effective to achieve its intended purpose. While individual needs vary, determination of optimal ranges of effective amounts of each component is within the skill of the art. Typically, a Compound of the Disclosure is administered in an amount from about 0.05 mg/kg to about 500 mg/kg, about 0.05 mg/kg to about 100 mg/kg, about 0.05 mg/kg to about 50 mg/kg, or about 0.05 mg/kg to about 10 mg/kg. The dosage of a composition can be at any dosage including, but not limited to, about 0.05 mg/week to about 100 mg/week. Particular doses include 0.05, 1, 2, 5, 10, 20, 500, and 100 mg/kg once daily, or once weekly. In one embodiment, a Compound of the Disclosure is administered one, two, three, four, or five times a week, i.e., the Compound of the Disclosure is administered according to an intermittent dosing schedule. These dosages are exemplary, but there can be individual instances in which higher or lower dosages are merited, and such are within the scope of this disclosure. In practice, the physician determines the actual dosing regimen that is most suitable for an individual patient, which can vary with the age, weight, and response of the particular patient.

The unit oral dose of a Compound of the Disclosure may comprise from about 0.01 to about 1000 mg, e.g., about 0.01 to about 100 mg of Compound of the Disclosure. In one embodiment, the unit oral dose of Compound of the Disclosure is 0.05 mg, 1 mg, 3 mg, 5 mg, 7 mg, 9 mg, 10 mg 12 mg, 14 mg, 15 mg, 17 mg, 20 mg, 22 mg, 25 mg, 27 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. The unit dose may be administered one or more times daily, e.g., as one or more tablets or capsules. The unit does may also be administered by IV or subcutaneously to the subject. In practice, the physician determines the actual dosing regimen that is most suitable for an individual patient, which can vary with the age, weight, and response of the particular patient.

In addition to administering a Compound of the Disclosure as a raw chemical, it may be administered as part of a pharmaceutical preparation or composition. In some embodiments, the pharmaceutical preparation or composition can include one or more pharmaceutically acceptable carriers, excipients, and/or auxiliaries. In some embodiments, the one or more carriers, excipients, and auxiliaries facilitate processing of a Compound of the Disclosure into a preparation or composition which can be used pharmaceutically. The preparations, particularly those preparations which can be administered orally, subcutaneously, or topically, and which can be used for one type of administration, such as tablets, dragees, slow release lozenges and capsules, mouth rinses and mouth washes, gels, liquid suspensions, hair rinses, hair gels, and shampoos, and also preparations which can be administered rectally, such as suppositories, as well as suitable solutions for administration by intravenous infusion, subcutaneous injection, topically or orally, contain from about 0.01 to 99 percent, in one embodiment from about 0.25 to 75 percent of active compound(s), together with the one or more carriers, excipients, and/or auxiliaries.

The compounds and pharmaceutical compositions provided herein may be administered to any subject which may experience the beneficial effects of a Compound of the Disclosure. Foremost among such subjects are mammals, e.g., humans, although the methods and compositions provided herein are not intended to be so limited. Other subjects include veterinary animals (cows, sheep, pigs, horses, dogs, cats and the like). In one embodiment, the subject is a human cancer patient.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers.

Therapeutically effective amounts of a Compound of the Disclosure and optional therapeutic agent(s) can be formulated in accordance with standard pharmaceutical practices, are administered to a human subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure and optional therapeutic agent(s) can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure and optional therapeutic agent(s) are administered in an effective amount to achieve its intended purpose. The exact formulation, route of administration, and dosage is determined by an individual physician in view of the diagnosed condition or disease. Dosage amount and interval can be adjusted individually to provide levels of Compound of the Disclosure and the optional therapeutic agent(s) that is sufficient to maintain therapeutic effects.

Toxicity and therapeutic efficacy of a Compound of the Disclosure and optional therapeutic agent(s) can be determined by standard pharmaceutical procedures in cell cultures or experimental animals, e.g., for determining the maximum tolerated dose (MTD) of a compound, which defines as the highest dose that causes no toxicity in a patient. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure and optional therapeutic agent(s) for use in therapy varies with the nature of the condition being treated, the length of time that activity is desired, and the age and the condition of the subject, and ultimately is determined by the attendant physician. For example, dosage amounts and intervals can be adjusted individually to provide plasma levels of a Compound of the Disclosure and/or optional therapeutic agent(s) that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: one dose per day; four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The optional therapeutic agent(s) is administered in therapeutically effective amounts. For example, when the optional therapeutic agent(s) is an immune checkpoint inhibitor, and the immune checkpoint inhibitor is a monoclonal antibody, 1-20 mg/kg is administered as an intravenous infusion every 2-4 weeks. For example, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg and 2000 mg of the antibody may be administered.

For example, when the immune checkpoint inhibitor is the anti-PD-1 antibody nivolumab, 3 mg/kg may be administered by intravenous infusion over 60 minutes every two weeks. When the immune checkpoint inhibitor is the anti-PD-1 antibody pembrolizumab, 2 mg/kg may be administered by intravenous infusion over 30 minutes every two or three weeks. When the immune checkpoint inhibitor is the anti-PD-L1 antibody avelumab, 10 mg/kg may be administered by intravenous infusion as frequently as every 2 weeks. Disis et al., J. Clin Oncol. 33 (2015) (suppl; abstr 5509). When the immune checkpoint inhibitor is the anti-PD-L1 antibody MPDL3280A, 20 mg/kg may be administered by intravenous infusion every 3 weeks. Herbst et al., Nature 515:563-80 (2014). When the immune checkpoint inhibitor is the anti-CTLA-4 antibody ipilumumab, 3 mg/kg may be administered by intravenous infusion over 90 minutes every 3 weeks. When the immune checkpoint inhibitor is the anti-CTLA-4 antibody tremelimumab, 15 mg/kg may be administered by intravenous infusion every 12 weeks. Naido et al., British Journal of Cancer 111:2214-19 (2014); Drugs R D, 10:123-32 (2010). When the immune checkpoint inhibitor is the anti-LAG3 antibody GSK2831781, 1.5 to 5 mg/kg may be administered by intravenous infusion over 120 minutes every 2-4 weeks. When the immune checkpoint inhibitor is an anti-TIM3 antibody, 1-5 mg/kg may be administered by intravenous infusion over 30-90 minutes every 2-4 weeks. When an inhibitor of indoleamine 2,3-dioxygenase (IDO) pathway is inhibitor indoximod in combination with temozolomide, 18.5 mg/kg/dose BID with an escalation to 27.7 mg/kg/dose BID of indoximod with 200 mg/m² every 5 days of temozolomide.

In one embodiment, the immune checkpoint inhibitor is an antibody and 1-20 mg/kg is administered by intravenous infusion every 2-4 weeks. In another embodiment, 50-2000 mg of the antibody is administered by intravenous infusion every 2-4 weeks. In another embodiment, a Compound of the Disclosure is administered prior to administration of the antibody. In another embodiment, a Compound of the Disclosure is administered 3-7 days prior to the day of administration of the antibody. In another embodiment, a Compound of the Disclosure is also administered the day the antibody is administered and on consecutive days thereafter until disease progression or until Compound of the Disclosure administration is no longer beneficial.

In one embodiment, the cancer patient receives 2 mg/kg pembrolizumab administered by intravenous infusion every three weeks and, e.g., about 0.1 to 100 mg of a Compound of the Disclosure administered for 1-7 days prior to pembrolizumab administration, optionally, on the day of pembrolizumab administration, and, optionally, thereafter until disease progression or until there is no therapeutic benefit.

In another embodiment, the cancer patient receives 3 mg/kg nivolumab administered by intravenous infusion every 2 weeks and, e.g., about 0.1 to 100 mg of a Compound of the Disclosure administered for 1-7 days prior to nivolumab administration, optionally, on the day of nivolumab administration, and, optionally, thereafter until disease progression or until there is no therapeutic benefit.

In another embodiment, the cancer patient receives 3 mg/kg nivolumab administered by intravenous infusion every 2 weeks and, e.g., about 0.1 to 100 mg of a Compound of the Disclosure administered for 1-7 days prior to nivolumab administration, optionally, on the day of nivolumab administration, and, optionally, thereafter until disease progression or until there is no therapeutic benefit.

In another embodiment, the treatment of the cancer patient with a Compound of the Disclosure and an immune checkpoint inhibitor induces anti-proliferative response faster than when the immune checkpoint inhibitor is administered alone.

In one embodiment, the disclosure provides a method of treating cancer in a subject, wherein the cancer is a solid tumor. In another embodiment, the cancer is a hematological cancer.

In another embodiment, the cancer is any one or more of the cancers of Table 3.

TABLE 3

| | | | |
|---|---|---|---|
| adrenal cancer | acinic cell | acoustic neuroma | acral |
| | carcinoma | | lentigious |
| | | | melanoma |
| acrospiroma | acute | acute | acute |
| | eosinophilic | erythroid | lymphoblastic |
| | leukemia | leukemia | leukemia |
| acute | acute | acute | adeno- |
| mega- | monocytic | promyelocytic | carcinoma |
| karyoblastic | leukemia | leukemia | |
| leukemia | | | |
| adenoid | adenoma | adenomatoid | adeno- |
| cystic | | odontogenic | squamous |
| carcinoma | | tumor | carcinoma |
| adipose | adrenocortical | adult T-cell | aggressive |
| tissue | carcinoma | leukemia/ | NK-cell |
| neoplasm | | lymphoma | leukemia |
| AIDS- | alveolar | alveolar | ameloblastic |
| related | rhabdomyo- | soft part | fibroma |
| lymphoma | sarcoma | sarcoma | |
| anaplastic | anaplastic | angio- | angio- |
| large cell | thyroid | immunoblastic | myolipoma |
| lymphoma | cancer | T-cell | |
| | | lymphoma | |
| angiosarcoma | astrocytoma | atypical | B-cell chronic |
| | | teratoid | lymphocytic |
| | | rhabdoid | leukemia |
| | | tumor | |
| B-cell | B-cell | basal cell | biliary |
| prolymphocytic | lymphoma | carcinoma | tract |
| leukemia | | | cancer |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid | clear-cell | cranio- |
| | plexus | sarcoma of | pharyngioma |
| | papilloma | the kidney | |
| cutaneous | cervical cancer | colorectal | Degos |
| T-cell | | cancer | disease |
| lymphoma | | | |
| desmoplastic | diffuse large | dysembryo- | dysgerminoma |
| small round | B-cell | plastic | |
| cell tumor | lymphoma | neuroepithelial | |
| | | tumor | |
| embryonal | endocrine | endodermal | enteropathy- |
| carcinoma | gland | sinus | associated |
| | neoplasm | tumor | T-cell |
| | | | lymphoma |
| esophageal | fetus in fetu | fibroma | fibrosarcoma |
| cancer | | | |
| follicular | follicular | ganglioneuroma | gastro- |
| lymphoma | thyroid | | intestinal |
| | cancer | | cancer |
| germ cell tumor | gestational | giant cell | giant cell |
| | chorio- | fibroblastoma | tumor of |
| | carcinoma | | the bone |
| glial tumor | glioblastoma | glioma | gliomatosis |
| | multiforme | | cerebri |
| glucagonoma | gonadoblastoma | granulosa | gynandro- |
| | | cell tumor | blastoma |
| gallbladder | gastric cancer | hairy cell | hemangio- |
| cancer | | leukemia | blastoma |
| head | hemangio- | hematological | hepato- |
| and neck | pericytoma | cancer | blastoma |
| cancer | | | |
| hepatosplenic | Hodgkin's | non-Hodgkin's | invasive |
| T-cell | lymphoma | lymphoma | lobular |
| lymphoma | | | carcinoma |
| intestinal | kidney cancer | laryngeal cancer | lentigo |
| cancer | | | maligna |
| lethal | leukemia | leydig | liposarcoma |
| midline | | cell tumor | |
| carcinoma | | | |
| lung cancer | lymphangioma | lymphangio- | lympho- |
| | | sarcoma | epithelioma |
| lymphoma | acute | acute | chronic |
| | lymphocytic | myelogeous | lymphocytic |
| | leukemia | leukemia | leukemia |
| liver cancer | small cell | non-small | MALT |
| | lung cancer | cell | lymphoma |
| | | lung cancer | |
| malignant | malignant | malignant | mantle cell |
| fibrous | peripheral | triton | lymphoma |
| histiocytoma | nerve | tumor | |
| | sheath tumor | | |
| marginal | mast cell | mediastinal | medullary |
| zone B-cell | leukemia | germ | carcinoma |
| lymphoma | | cell tumor | of the |
| | | | breast |
| medullary | medulloblastoma | melanoma | meningioma |
| thyroid | | | |
| cancer | | | |
| merkel | mesothelioma | metastatic | mixed |
| cell cancer | | urothelial | Mullerian |
| | | carcinoma | tumor |
| mucinous tumor | multiple myeloma | muscle | mycosis |
| | | tissue | fungoides |
| | | neoplasm | |
| myxoid | myxoma | myxosarcoma | naso- |
| liposarcoma | | | pharyngeal |
| | | | carcinoma |
| neurinoma | neuroblastoma | neurofibroma | neuroma |
| nodular | ocular cancer | oligoastro- | oligo- |
| melanoma | | cytoma | dendroglioma |
| oncocytoma | optic nerve | optic | oral cancer |
| | sheath | nerve tumor | |
| | meningioma | | |
| osteosarcoma | ovarian cancer | Pancoast tumor | papillary |
| | | | thyroid |
| | | | cancer |
| paraganglioma | pinealoblastoma | pineocytoma | pituicytoma |
| pituitary | pituitary tumor | plasmacytoma | poly- |
| adenoma | | | embryoma |
| precursor T- | primary | primary | preimary |
| lymphoblastic | central | effusion | peritoneal |
| lymphoma | nervous | lymphoma | cancer |
| | system | | |
| | lymphoma | | |
| prostate cancer | pancreatic cancer | pharyngeal | pseudo- |
| | | cancer | myxoma |
| | | | periotonei |
| renal cell | renal | retinoblastoma | rhabdomyoma |
| carcinoma | medullary | | |
| | carcinoma | | |
| rhabdo- | Richter's | rectal cancer | sarcoma |
| myosarcoma | transformation | | |
| Schwanno- | seminoma | Sertoli cell tumor | sex cord- |
| matosis | | | gonadal |
| | | | stromal tumor |
| signet ring cell | skin cancer | small blue | small cell |
| carcinoma | | round cell | carcinoma |
| | | tumors | |
| soft tissue | somatostatinoma | soot wart | spinal tumor |
| sarcoma | squamous cell | synovial sarcoma | Sezary's |
| splenic | carcinoma | | disease |
| marginal | | | |
| zone | | | |
| lymphoma | | | |
| small | squamous | stomach cancer | T-cell |
| intestine | carcinoma | | lymphoma |
| cancer | | | |
| testicular | thecoma | thyroid cancer | transitional |
| cancer | | | cell |
| | | | carcinoma |
| throat cancer | urachal cancer | urogenital cancer | urothelial |
| | | | carcinoma |
| uveal melanoma | uterine cancer | verrucous | visual |
| | | carcinoma | pathway |
| | | | glioma |
| vulvar cancer | vaginal cancer | Waldenstrom's | Warthin's |
| | | macro- | tumor |
| | | globulinemia | |
| Wilms' tumor | | | |

Exemplary hematological cancers include, but are not limited to, the cancers listed in Table 4. In another embodiment, the hematological cancer is acute lymphocytic leukemia, chronic lymphocytic leukemia (including B-cell chronic lymphocytic leukemia), or acute myeloid leukemia.

TABLE 4

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's | T-cell lymphoma |
| macroglobulinemia (WM) | |
| diffuse large B-cell | mast cell leukemia |
| lymphoma (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic |
| | T-cell lymphoma |
| Richter's transformation | |

In another embodiment, the cancer is selected from the group consisting of squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, and squamous cell carcinoma of the skin.

In another embodiment, a precancerous tumor is selected from the group consisting of leukoplakia of the head and neck, Barrett's esophagus, metaplasia of the stomach, adenoma of the colon, chronic hepatitis, bile duct hyperplasia, pancreatic intraepithelial neoplasia, atypical adenomatous hyperplasia of the lungs, dysplasia of the bladder, cervical initraepithelial neoplasia, penile intraepithelial neoplasia, and actinic keratosis of the skin.

In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the cancer is selected from the group consisting of colorectal cancer, breast cancer, lymphoma, melanoma, kidney cancer, and lung cancer.

In another embodiment, the cancer has become resistant to conventional cancer treatments. The term “conventional cancer treatments” as used herein refers to any cancer drugs, biologics, or radiotherapy, or combination of cancer drugs and/or biologics and/or radiotherapy that have been tested and/or approved for therapeutic use in humans by the U.S. Food and Drug Administration, European Medicines Agency, or similar regulatory agency.

In another embodiment, the subject has been treated previously with an anticancer agent, e.g., an immune checkpoint inhibitor, without a Compound of the Disclosure. For example, the previous immune checkpoint therapy may be an anti-PD-1 or anti-PD-L1 therapy.

In another embodiment, the present disclosure provides therapeutic methods of treating a subject having cancer, comprising administering to the subject a therapeutically effective amount of a Compound of the Disclosure, wherein the Compound of the Disclosure is administered to the subject according to an intermittent dosing schedule.

In another embodiment, the present disclosure provides therapeutic methods of treating a subject having cancer, comprising administering to the subject therapeutically effective amounts of a Compound of the Disclosure, and an optional therapeutic agent, e.g., an immune checkpoint inhibitor, wherein the Compound of the Disclosure is administered to the subject according to an intermittent dosing schedule.

In another embodiment, the present disclosure provides therapeutic methods of treating a subject having cancer, comprising administering to the subject therapeutically effective amounts of a Compound of the Disclosure, an immune checkpoint inhibitor, and a third optional therapeutic agent, e.g., radiation.

In another embodiment, the present disclosure provides kits comprising a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

The present disclosure provides the following particular embodiments with regard to therapeutic methods.

Embodiment I. A method of treating cancer a subject in need thereof, the method comprising administering to the subject a therapeutically effective amount of the a Compound of the Disclosure.

Embodiment II. The method of Embodiment I, wherein the cancer is a solid tumor.

Embodiment III. The method of Embodiment I, wherein the cancer is a hematological cancer.

Embodiment IV. The method of Embodiment I, wherein the cancer is any one or more of the cancers of Table 3.

Embodiment V. The method of Embodiment I, wherein the cancer is any one or more of the cancers of Table 4.

Embodiment VI. The method of any one of Embodiments I-V further comprising administering to the subject a therapeutically effective amount of one or more optional therapeutic agents useful in the treatment of cancer.

Embodiment VII. The pharmaceutical composition comprising a Compound of the Disclosure and one or more pharmaceutically acceptable excipients for use in treating cancer.

Embodiment VIII. The pharmaceutical composition of Embodiment VII, wherein the cancer is a solid tumor.

Embodiment IX. The pharmaceutical composition of Embodiment VII, wherein the cancer is a hematological cancer.

Embodiment X The pharmaceutical composition of Embodiment VII, wherein the cancer is any one or more of the cancers of Table 3.

Embodiment XI. The pharmaceutical composition of Embodiment VII, wherein the cancer is any one or more of the cancers of Table 4.

Embodiment XII. A Compound of the Disclosure for use in treating of cancer.

Embodiment XIII. The compound for use of Embodiment XII, wherein the cancer is a solid tumor.

Embodiment XIV. The compound for use of Embodiment XII, wherein the cancer is a hematological cancer.

Embodiment XV The compound for use of Embodiment XII, wherein the cancer is any one or more of the cancers of Table 3.

Embodiment XVI. The compound for use of Embodiment XII, wherein the cancer is any one or more of the cancers of Table 4.

Embodiment XVII. The compound for use of any one of Embodiments XII-XVI, wherein the compound is to be administered to the subject in combination with a therapeutically effective amount of one or more optional therapeutic agents useful in the treatment of cancer.

Embodiment XVIII. Use of a Compound of the Disclosure for the manufacture of a medicament for treatment of cancer.

Embodiment XIX. The use of Embodiment XVIII, wherein the cancer is a solid tumor.

Embodiment XX. The use of Embodiment XVIII, wherein the cancer is a hematological cancer.

Embodiment XXI The use of Embodiment XVIII, wherein the cancer is any one or more of the cancers of Table 3.

Embodiment XXII. The use of Embodiment XVIII, wherein the cancer is any one or more of the cancers of Table 4.

Embodiment XXIII. The use of any one of Embodiments XVIII-XXII, wherein the compound is to be administered to the in combination with a therapeutically effective amount of one or more optional therapeutic agents useful in the treatment of cancer.

Embodiment XXIV. A therapeutic or prophylactic agent for cancer, which comprises a Compound of the Disclosure.

Embodiment XXV. A kit comprising a Compound of the Disclosure and instructions for administering the compound to a subject having cancer.

Embodiment XXVI. The kit of Embodiment XXV, wherein the cancer is a solid tumor.

Embodiment XXVII. The kit of Embodiment XXV, wherein the cancer is a hematological cancer.

Embodiment XXVIII. The kit of Embodiment XXV, wherein the cancer is any one or more of the cancers of Table 3.

Embodiment XXIX. The kit of Embodiment XXV, wherein the cancer is any one or more of the cancers of Table 4.

Embodiment XXX. The kit of any one of Embodiments XXV-XXIX further comprising one or more optional therapeutic agents useful in the treatment of cancer.

### III. Optional Therapeutic Agents

In some therapeutic methods and uses of the disclosure, a Compound of the Disclosure is administered to a subject having a disease, disorder, or condition, e.g., cancer, as a single agent. In other therapeutic methods and uses of the disclosure, a Compound of the Disclosure is administered to a subject having a disease, disorder, or condition, e.g., cancer, in combination with one or more optional therapeutic agents. In one embodiment, a Compound of the Disclosure is administered in combination with one optional therapeutic agent. In another embodiment, a Compound of the Disclosure is administered in combination with two optional therapeutic agents. In another embodiment, a Compound of the Disclosure is administered in combination with three optional therapeutic agents. Optional therapeutic agents useful in treating cancer patients include those known in the art as well as those developed in the future.

Optional therapeutic agents are administered in an amount to provide their desired therapeutic effect. The effective dosage range for each optional therapeutic agent is known in the art, and the optional therapeutic agent is administered to an individual in need thereof within such established ranges.

A Compound of the Disclosure and the optional therapeutic agent(s) can be administered together as a single-unit dose or separately as multi-unit doses, and in any order, e.g., wherein a Compound of the Disclosure is administered before the optional therapeutic agent(s), or vice versa. One or more doses of a Compound of the Disclosure and the optional therapeutic agent(s) can be administered to the subject.

In one embodiment, the optional therapeutic agent is an immune checkpoint inhibitor. Immune checkpoint inhibitors are therapies that blockade immune system inhibitor checkpoints. Immune checkpoints can be stimulatory or inhibitory. Blockade of inhibitory immune checkpoint activates immune system function and can be used for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053 and have the structure.

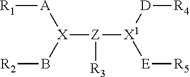

or a pharmaceutically acceptable salt thereof, wherein the compound comprises at least 5 amino acids useful as therapeutic agents capable of inhibiting the PD-1 signaling pathway.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Lob et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In another embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, the optional therapeutic agent is a chemotherapeutic agent or other anti-proliferative agent that can be administered in combination with a Compound of the Disclosure to treat cancer. Examples of conventional therapies and anticancer agents that can be used in combination with a Compound of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Nonlimiting exemplary topoisomerase I inhibitors include topotecan, gimatecan, irnnotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include famesyl transferase inhibitors, such as L-744832, DK8G557, tipifarnib, and lonafamib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib.

Nonlimiting exemplary FMS-like tyrosine kinase inhibitors, which are compounds targeting, decreasing or inhibiting the activity of FMS-like tyrosine kinase receptors (Flt-3R) include interferon, I-β-D-arabinofuransylcytosine (ara-c), and bisulfan; and ALK inhibitors, which are compounds which target, decrease, or inhibit anaplastic lymphoma kinase.

Nonlimiting exemplary Flt-3 inhibitors include PKC412, midostaurin, a staurosporine derivative, SU11248, and MLN518.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a famesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, osimertinib, OSI-774, Cl-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, anticancer agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor. Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a subject having cancer (a cancer patient) therapeutically effective amounts of a Compound of the Disclosure, an immune checkpoint inhibitor, and at least one additional optional therapeutic agent, e.g., an anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUCI-IL2, nilutamide, nitrocamptothecin, nolatrexed dihydrochlonide, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R15777, RAD001, ranpimase, rebeccamycin analogue, rhuAngiostatin protein, rhuMab 2C4, rosiglitazone, rubitecan, S-1, 5-8184, satraplatin, SB-, 15992, SGN-0010, SGN-40, sorafenib, SR31747A, ST1571, SU01248, suberoylanilide hydroxamic acid, suramin, talabostat, talampanel, tariquidar, temsirolimus, TGFa-P38 immunotoxin, thalidomide, thymalfasin, tipifarmib, tirapazamine, TLK286, trabectedin, trimetrexate glucuronate, TroVax, UCN-1, valproic acid, vinflunine, VNP4M, volociximab, vorinostat, VX-680, ZD1839, ZD6474, zileuton, and zosuquidar trihydrochHoride.

In one embodiment, the optional therapeutic agent comprises one of the anti-cancer drugs or anti-cancer drug combinations listed in Table 5.

TABLE 5

| | | | |
|---|---|---|---|
| Abemaciclib | Abiraterone | Abraxane (Paclitaxel | ABVD |
| | Acetate | Albumin-stabilized | |
| | | Nanoparticle | |
| | | Formulation) | |
| ABVE | ABVE-PC | AC | Acalabrutinib |
| AC-T | Actemra | Adcetris (Brentilximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S-Malate) |
| Cabozantinib-S-Malate | CAF | Calquence | Campath |
| | | (Acalabrutinib) | (Alemtuzumab) |
| Camptosar | Capecitabine | CAPOX | Carac |
| (Irinotecan | | | (Fluorouracil-Topical) |
| Hydrochloride) | | | |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-Topical) | Acetate) | (Rasburicase) |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cettlximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil- | | (Panobinostat) |
| | Topical) | | |
| Faslodex | FEC | Femara (Letrozole) | Filgrastim |
| (Fulvestrant) | | | |
| Firmagon | Fludarabine | Fluoroplex | Fluorouracil |
| (Degarelix) | Phosphate | (Fluorouracil-Topical) | Injection |
| Fluorouracil- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva | Gefitinib |
| (Recombinant | (Recombinant | (Obinutuzumab) | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa-2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan Kerastik | Libtayo |
| (Chlorambucil) | Acetate | (Aminolevulinic Acid) | (Cemiplimab-rwlc) |
| LipoDox | Lomustine | Lonsurf | Lorbrena |
| (Doxorubicin | | (Trifluridine and | (Lorlatinib) |
| Hydrochloride | | Tipiracil | |
| Liposome) | | Hydrochloride) | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza | Marqibo |
| (Lutetium Lu 177- | Dotatate) | (Olaparib) | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin | Prolia |
| Hydrochloride | Alfa) | (Aldesleukin) | (Denosumab) |
| Promacta | Propranolol | Provenge | Purinethol |
| (Eltrombopag | Hydrochloride | (Sipuleucel-T) | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant | Regorafenib |
| Human | Human | Interferon | |
| Papillomavirus | Papillomavirus | Alfa-2b | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rittman Hycela | Rituximab | Rituximab and | Rolapitant |
| (Rituximab and | | Hyaluronidase | Hydrochloride |
| Hyaluronidase | | Human | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard | Vitrakvi |
| | | (Uridine | (Larotrectinib |
| | | Triacetate) | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda | XELIRI |
| (Daunorubicin | (Crizotinib) | (Capecitabine) | |
| Hydrochloride and | | | |
| Cytarabine | | | |
| Liposome) | | | |
| XELOX | Xgeva | Xofigo | Xtandi |
| | (Denosumab) | (Radium 223 | (Enzalutamide) |
| | | Dichloride) | |
| Yervoy | Yescarta | Yondelis (Trabectedin) | Zaltrap |
| (Ipilimumab) | (Axicabtagene | | (Ziv-Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran | Zoladex |
| (Dexrazoxane | | (Ondansetron | (Goserelin |
| Hydrochloride) | | Hydrochloride) | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, methods provided herein comprise administering a Compound of the Disclosure in combination with radiation therapy. The methods provided herein are not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### IV. Biomarkers

In another embodiment, present disclosure provides methods of treating a subject having cancer comprising administering a therapeutically effective amount of a Compound of the Disclosure to the subject if a biomarker is present in a biological sample of the subject. In another embodiment, the method comprises determining whether a biomarker is present or absent in the biological sample. See, e.g., Goossens et al., Transl Cancer Res. 4:256-269 (2015); Kamel and Al-Amodi, Genomics Proteomics Bioinformatics 15:220-235 (2017); and Konikova and Kusenda, Neoplasma 50:31-40 (2003).

The term “biomarker” as used herein refers to any biological compound, such as a gene, a protein, a fragment of a protein, a peptide, a polypeptide, a nucleic acid, etc., or chromosome abnormality, such as a chromosome translocation, that can be detected and/or quantified in a cancer patient in vivo or in a biological sample obtained from a cancer patient. A biomarker can be the entire intact molecule, or it can be a portion or fragment thereof. In one embodiment, the expression level of the biomarker is measured. The expression level of the biomarker can be measured, for example, by detecting the protein or RNA, e.g., mRNA, level of the biomarker. In some embodiments, portions or fragments of biomarkers can be detected or measured, for example, by an antibody or other specific binding agent. In some embodiments, a measurable aspect of the biomarker is associated with a given state of the patient, such as a particular stage of cancer. For biomarkers that are detected at the protein or RNA level, such measurable aspects may include, for example, the presence, absence, or concentration, i.e., expression level, of the biomarker in a cancer patient, or biological sample obtained from the cancer patient. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes. In one embodiment, the measurable aspect of the biomarker is its expression status. In one embodiment, the measurable aspect of the biomarker is its mutation status.

In one embodiment, the biomarker is the mutation status of any one or more of BRAF, KRAS, p53, and/or PI3KCA, which is differentially present in a subject of one phenotypic status, e.g., a subject having a hematological cancer, as compared with another phenotypic status, e.g., a normal undiseased subject or a patient having cancer without a mutation in BRAF, KRAS, etc. Methods to detect mutations in these biomarkers are known in the art.

In another embodiment, the biomarker is the expression status of MYC which is differentially present in a subject of one phenotypic status, e.g., a subject having a hematological cancer, as compared with another phenotypic status, e.g., a normal undiseased subject or a patient having cancer without an overexpression of MYC. Methods the expression status of MYC are known in the art.

In another embodiment, the biomarker is the mutation status of BRAF, KRAS, or both, which is differentially present in a subject of one phenotypic status, e.g., a subject having a hematological cancer, as compared with another phenotypic status, e.g., a normal undiseased subject or a patient having cancer without a mutation in BRAF, KRAS, or both. Methods to detect mutations in BRAF and KRAS are known in the art. See, e.g., Loes et al., Tumour Biol. 36(2): 1003-1013 (2015).

Biomarker standards can be predetermined, determined concurrently, or determined after a biological sample is obtained from the subject. Biomarker standards for use with the methods described herein can, for example, include data from samples from subjects without cancer; data from samples from subjects with cancer, e.g., breast cancer, that is not metastatic; and data from samples from subjects with cancer, e.g., breast cancer, that metastatic. Comparisons can be made to establish predetermined threshold biomarker standards for different classes of subjects, e.g., diseased vs. non-diseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., a cancer patient, belongs to one phenotypic status or another.

In addition to individual biological compounds, e.g., BRAF or KRAS, the term “biomarker” as used herein is meant to include groups, sets, or arrays of multiple biological compounds. For example, the combination of BRAF and KRAS mutation status may comprise a biomarker. The term “biomarker” may comprise one, two, three, four, five, six, seven, eight, nine, ten, fifteen, twenty, twenty five, thirty, or more, biological compounds.

The determination of the expression level or mutation status of a biomarker in a patient can be performed using any of the many methods known in the art. Any method known in the art for quantitating specific proteins and/or detecting BRAF and/or KRAS mutation status, or the expression or mutation levels of any other biomarker in a patient or a biological sample may be used in the methods of the disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, N.Y., (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample is obtained from the patient and the biological sample is assayed for determination of a biomarker expression or mutation status.

In another embodiment of the disclosure, Northern blot analysis of biomarker transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabled probes and their use are well known in the art. The presence and or relative levels of expression of the biomarker being assayed can be quantified using, for example, densitometry.

In another embodiment, biomarker expression and/or mutation status is determined using RT-PCR. RT-PCR allows detection of the progress of a PCR amplification of a target gene in real time. Design of the primers and probes required to detect expression and/or mutation status of a biomarker of the disclosure is within the skill of a practitioner of ordinary skill in the art. RT-PCR can be used to determine the level of RNA encoding a biomarker of the disclosure in a tumor tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5′ end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In another embodiment of the disclosure, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In another embodiment of the disclosure, the expression of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

The RAF kinases (A-RAF, BRAF, and C-RAF) are key components of the mitogen-activated protein kinase (MAPK) pathway that controls cell proliferation and survival signaling. (Downward, Nature Reviews Cancer 3(1):11-22 (2003); Wellbrock et al., Nature Reviews Molecular Cell Biology 5(11):875-85 (2004). The MAP kinase (MAPK) pathway is a central signal transduction pathway that is dysregulated in a large number of developmental disorders. The MAPK pathway, which is composed of RAS, RAF, MAPK or extracellular signal-regulated kinase (MEK), and extracellular signal-regulated kinase (ERK), integrates signals from receptors on the cell surface including cancer-related receptor tyrosine kinases such as the epidermal growth factor receptor, mesenchymal-epithelial transition factor (MET), and vascular endothelial growth factor receptor (Avruch, Biochim Biophys Acta 1773(8):1150-60 (2007). Genetic alterations in the MAPK pathway are among the most common in human cancers. Up to 60% of melanomas harbor BRAF mutations (Davies et al., Nature 417:949-54 (2002)) and KRAS mutations have been estimated in roughly 60%, 30%, and 15% of pancreatic, colon, and lung tumors, respectively (Vakiani et al., J Pathol 223(2):219-29 (2011). BRAF mutations are also found in 40% of papillary or anaplastic thyroid cancers (Kimura et al., Cancer Res 63(7):1454-7 (2003) and in a small percentage of several other types of tumor (Vakiani et al., J Pathol 223(2):219-29 (2011). A majority of reported BRAF mutations are a substitution of glutamic acid for valine at the amino acid position of 600 (the V600E mutation). The BRAF V600E mutation constitutively activates BRAF and downstream signal transduction in the MAPK pathway (Davies et al., Nature 417:949-54 (2002).

In one embodiment, the disclosure provides a method of treating a subject having cancer, the method comprising administering a therapeutically effective amount of a Compound of the Disclosure to the subject if a mutation in BRAF, KRAS, p53, and/or PI3KCA, is present in a biological sample of the subject. In another embodiment, the method comprises determining whether a mutation in BRAF, KRAS, p53, and/or PI3KCA, is present or absent in the biological sample.

In another embodiment, the disclosure provides a method of identifying whether a subject having cancer as a candidate for treatment with a Compound of the Disclosure, the method comprising:

(a) identifying the subject as being a candidate for treatment if a mutation in BRAF, KRAS, p53, and/or PI3KCA, is present; or

(b) identifying the subject as not being a candidate for treatment if a mutation in BRAF, KRAS, p53, and/or PI3KCA, is absent. In another embodiment, the method comprises determining whether a mutation in BRAF, KRAS, p53, and/or PI3KCA, is present or absent in the biological sample.

In another embodiment, the disclosure provides a method of predicting treatment outcome in a subject having cancer, the method comprising:

(a) if there is a mutation in BRAF, KRAS, p53, and/or PI3KCA, in the biological sample, then administering a Compound of the Disclosure to the subject will likely cause a favorable therapeutic response; and

(b) if there is an absence of a mutation in BRAF, KRAS, p53, and/or PI3KCA, in the biological sample, then administering a Compound of the Disclosure to the subject will likely cause an unfavorable therapeutic response. In another embodiment, the method comprises determining whether a mutation in BRAF, KRAS, p53, and/or PI3KCA, is present or absent in the biological sample.

In another embodiment, the disclosure provides a method, comprising administering a therapeutically effective amount of a Compound of the Disclosure to a subject in need thereof, wherein:

(a) the subject has cancer; and

(b) the cancer is characterized as having a mutation in BRAF, KRAS, p53, and/or PI3KCA.

In another embodiment, the disclosure provides any of the above biomarker-related methods, wherein the mutation is a mutation in BRAF. In another embodiment, the mutation in BRAF is a V600E mutation.

In another embodiment, the disclosure provides a method of treating a subject having cancer, the method comprising administering a therapeutically effective amount of a Compound of the Disclosure to the subject if an overexpression of MYC is present in a biological sample of the subject. In another embodiment, the method comprises determining whether an overexpression of MYC is present or absent in the biological sample.

In another embodiment, the disclosure provides a method of identifying whether a subject having cancer as a candidate for treatment with a Compound of the Disclosure, the method comprising:

(a) identifying the subject as being a candidate for treatment if an overexpression of MYC is present; or

(b) identifying the subject as not being a candidate for treatment if an overexpression of MYC is absent. In another embodiment, the method comprises determining whether an overexpression of MYC is present or absent in the biological sample.

In another embodiment, the disclosure provides a method of predicting treatment outcome in a subject having cancer, the method comprising:

(a) if there is an overexpression of MYC in the biological sample, then administering a Compound of the Disclosure to the subject will likely cause a favorable therapeutic response; and

(b) if there is an absence of an overexpression of MYC in the biological sample, then administering a Compound of the Disclosure to the subject will likely cause an unfavorable therapeutic response. In another embodiment, the method comprises determining whether an overexpression of MYC is present or absent in the biological sample.

In another embodiment, the disclosure provides a method, comprising administering a therapeutically effective amount of a Compound of the Disclosure to a subject in need thereof, wherein:

(a) the subject has cancer; and

(b) the cancer is characterized as having an overexpression of MYC.

### V. Definitions

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number ±10%. Thus, “about 10” means 9 to 11.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. However, in one embodiment, administration of a Compound of the Disclosure to a subject, with or without one or more optional therapeutic agents, leads to remission of the cancer.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase subject survival time by at least about 2%. In another embodiment, the therapeutic response is at least about 5%. In another embodiment, the therapeutic response is at least about 10%. In another embodiment, the therapeutic response is at least about 15%. In another embodiment, the therapeutic response is at least about 20%. In another embodiment, the therapeutic response is at least about 25%. In another embodiment, the therapeutic response is at least about 30%. In another embodiment, the therapeutic response is at least about 35%. In another embodiment, the therapeutic response is at least about 40%. In another embodiment, the therapeutic response is at least about 45%. In another embodiment, the therapeutic response is at least about 50%. In another embodiment, the therapeutic response is at least about 55%. In another embodiment, the therapeutic response is at least about 60%. In another embodiment, the therapeutic response is at least about 65%. In another embodiment, the therapeutic response is at least about 70%. In another embodiment, the therapeutic response is at least about 75%. In another embodiment, the therapeutic response is at least about 80%. In another embodiment, the therapeutic response is at least about 85%. In another embodiment, the therapeutic response is at least about 90%. In another embodiment, the therapeutic response is at least about 95%. In another embodiment, the therapeutic response is or at least about 100%, or more.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

In some embodiments, when administered in combination, two or more agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between a Compound of the Disclosure and the optional therapeutic agent, e.g., an immune checkpoint inhibitor, that causes the total effect of the drugs to be greater than the sum of the individual effects of each drug. See, e.g. Berenbaum, Pharmacological Reviews 41:93-141 (1989).

Synergy can be expressed in terms of a “Synergy Index (SI),” which is determined by the method described by F. C. Kull et al., Applied Microbiology 9:538 (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

The term “halo” as used herein by itself or as part of another group refers to —Cl, —F, —Br, or —I.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₆ alkyl. In another embodiment, the alkyl is a C₁-C₄ alkyl, i.e., methyl, ethyl, propyl, isopropyl, n-butyl, t-butyl, or iso-butyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “alkenyl” as used herein by itself or as part of another group refers to an alkyl group containing one or two carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “alkynyl” as used herein by itself or as part of another group refers to an alkyl group containing one carbon-to-carbon triple bond. In one embodiment, the alkynyl is a C₂-C₆ alkynyl. In another embodiment, the alkynyl is a C₂-C₄ alkynyl. Non-limiting exemplary alkynyl groups include ethynyl, propynyl, butynyl, 2-butynyl, pentynyl, and hexynyl groups.

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used herein by itself or as part of another group refers to an alkyl group attached to a terminal oxygen atom. In one embodiment, the alkyl is a C₁-C₆ alkyl. In another embodiment, the alkyl is a C₁-C₄ alkyl group. Non-limiting exemplary alkoxy groups include methoxy, ethoxy, and tert-butoxy.

The term “amino” as used by itself or as part of another group refers to a radical of the formula —NR^{a1}R^{a2}, wherein Rₐ₁ and Rₐ₂ are independently hydrogen, cycloalkyl, optionally substituted heterocyclo, optionally substituted aryl, optionally substituted heteroaryl, or (aryl)alkyl; or R^{a1} and R^{a2} are taken together with the nitrogen atom to which they are attached form a 4- to 7-membered optionally substituted heterocyclo. Non-limiting exemplary amino groups include —NH₂, —NH(CH₃), and —N(CH₃)₂.

The term “hydroxyalkyl” as used herein by itself or as part of another group refers to an alkyl group substituted with one or two hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary hydroxyl alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “carboxamido” as used herein by itself or as part of another group refers to a radical of formula —C(═O)NR^{b1}R^{b2}, wherein R^{b1} and R^{b2} are independently hydrogen, cycloalkyl, optionally substituted heterocyclo, optionally substituted aryl, optionally substituted heteroaryl, or (aryl)alkyl; or R^{b1} and R^{b2} are taken together with the nitrogen atom to which they are attached form a 4- to 7-membered optionally substituted heterocyclo. A non-limiting exemplary amino groups is —C(═O)NH₂.

The term “sulfonamido” as used herein by itself or as part of another group refers to a radical of formula —S(═O)₂NR^{c1}R^{c2}, wherein R^{c1} and R^{c2} are independently hydrogen, cycloalkyl, optionally substituted heterocyclo, optionally substituted aryl, optionally substituted heteroaryl, or (aryl)alkyl; or Rc^{a1} and R^{c2} are taken together with the nitrogen atom to which they are attached form a 4- to 7-membered optionally substituted heterocyclo. A non-limiting exemplary amino groups is —S(═O)₂NH₂.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “alkylsulfonyl” as used herein by itself or as part of another group refers to a sulfonyl group, i.e., —SO₂—, substituted by an alkyl group. In one embodiment, the alkyl is a C₁-C₄ alkyl. A non-limiting exemplary alkylsulfonyl group is —SO₂CH₃.

The term “alkoxyalkyl” as used herein by itself or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “(amino)alkyl” as used herein by itself or as part of another group refers to an alkyl substituted with one amino group. In one embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, and —CH₂CH₂N(CH₃)₂.

The term “(cyano)alkyl” as used herein by itself or as part of another group refers to an alkyl substituted with one cyano group. In one embodiment, the alkyl is a C₁-C₆ alkyl. In another embodiment, the alkyl is a C₁-C₄ alkyl. Non-limiting exemplary (cyano)alkyl groups include —CH₂CH₂CN and —CH₂CH₂CH₂CN.

The term “(carboxamido)alkyl” as used herein by itself or as part of another group refers to an alkyl substituted with a carboxamido group. In one embodiment, the alkyl is a C₁-C₄ alkyl. In another embodiment, the alkyl is a C₁ or C₂ alkyl. Non-limiting exemplary (carboxamido)alkyl groups include —CH₂C(═O)NH₂ and —CH₂C(═O)N(CH₃)₂.

The term “haloalkoxy” as used herein by itself or as part of another group refers to an haloalkyl group attached to a terminal oxygen atom. In one embodiment, the haloalkyl is a C₁-C₄ haloalkyl group. A non-limiting exemplary haloalkoxy group is —OCF₃.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”) and naphthyl, In one embodiment, the aryl group is phenyl.

The term “optionally substituted aryl” as used herein by itself or as part of another group refers to aryl that is either unsubstituted or substituted with one, two, three, four, or five substituents, wherein the substituents are each independently halo, nitro, cyano, hydroxy, amino, e.g., —NH₂, alkyl, haloalkyl, hydroxyalkyl, alkoxy, haloalkoxy, carboxamido, sulfonamido, alkylcarbonyl, alkylsulfonyl, alkoxyalkyl, (amino)alkyl, (cyano)alkyl, or (carboxamido)alkyl.

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having 5 to 14 ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl (a 5-membered heteroaryl having four carbon atoms and one sulfur atom). In another embodiment, the heteroaryl has 6 ring atoms, e.g., pyridyl (a 6-membered heteroaryl having five carbon atoms and one nitrogen atom). Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, D-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one, two, three, or four substituents, wherein the substituents are each independently halo, nitro, cyano, hydroxy, amino, e.g., —NH₂, alkyl, haloalkyl, hydroxyalkyl, alkoxy, haloalkoxy, carboxamido, sulfonamido, alkylcarbonyl, alkylsulfonyl, alkoxyalkyl, (amino)alkyl, (cyano)alkyl, or (carboxamido)alkyl. In one embodiment, the optionally substituted heteroaryl has two substituents. In another embodiment, the optionally substituted heteroaryl has one substituent. Any available carbon or nitrogen atom can be substituted.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group is either unsubstituted or substituted with one, two, or three substituents, wherein the substituents are each independently halo, nitro, cyano, hydroxy, amino, e.g., —NH₂, alkyl, haloalkyl, hydroxyalkyl, alkoxy, haloalkoxy, carboxamido, sulfonamido, alkylcarbonyl, alkylsulfonyl, alkoxyalkyl, (amino)alkyl, (cyano)alkyl, or (carboxamido)alkyl. In one embodiment, the optionally substituted cycloalkyl has two substituents. In another embodiment, the optionally substituted cycloalkyl has one substituent.

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzol[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom.

The term “optionally substituted heterocyclo” as used herein by itself or part of another group refers to a heterocyclo group that is either unsubstituted or substituted with one, two, three, or four substituents, wherein the substituents are each independently halo, nitro, cyano, hydroxy, amino, e.g., —NH₂, alkyl, haloalkyl, hydroxyalkyl, alkoxy, haloalkoxy, carboxamido, sulfonamido, alkylcarbonyl, alkylsulfonyl, alkoxyalkyl, (amino)alkyl, (cyano)alkyl, or (carboxamido)alkyl. In one embodiment, the optionally substituted heterocyclo has two substituents. In another embodiment, the optionally substituted heterocyclo has one substituent Substitution may occur on any available carbon or nitrogen atom of the heterocyclo group.

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure may contain one or more chiral centers and thus may give rise to enantiomers, diastereomers, and other stereoisomers. The present disclosure encompasses the use of all possible stereoisomeric forms of a Compound of the Disclosure, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

In one embodiment, Compounds of the Disclosure having one or more chiral centers are enantiomerically enriched, e.g., the ee is about 5% or more. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

The term “disease” or “condition” or “disorder” denotes disturbances and/or anomalies that as a rule are regarded as being pathological conditions or functions, and that can manifest themselves in the form of particular signs, symptoms, and/or malfunctions. Compounds of the Disclosure inhibit amino acid, e.g., glutamine, transporters, e.g., ASCT2, and can be used in treating diseases and conditions such as cancer and proliferative diseases, wherein inhibition of an amino acid, e.g., glutamine, transporter, e.g., ASCT2, provides a benefit.

The term “amino acid transporter” and the like as used herein refer membrane transport proteins that transport amino acids including, but not limited to glutamine, across the cell membrane. Amino acid transporters are well known in the art.

The terms “glutamine transporter” or “glutamine transport protein” and the like as used herein refer membrane transport proteins that transport glutamine across the cell membrane. ASCT2 and other glutamine transporters are well known in the art. For example, the sodium-dependent neutral amino acid transporter or “BOAT1” is a membrane transport protein encoded by the SLC6A19 gene. The sodium-coupled neutral amino acid transporter 1 or “SNAT1” is a membrane transport protein encoded by the SLC38A1 gene. The sodium-coupled neutral amino acid transporter 2 or “SNAT” is a membrane transport protein encoded by the SLC38A2 gene. The sodium-coupled neutral amino acid transporter 3 or “SNAT3” is a membrane transport protein encoded by the SLC38A3 gene. The sodium-coupled neutral amino acid transporter 5 or “SNAT5” is a membrane transport protein encoded by the SLC38A5 gene. The sodium-coupled neutral amino acid transporter 7 or “SNAT7” is a membrane transport protein encoded by the SLC38A7 gene. The large neutral amino acids transporter small subunit 1 or “LAT1” is a membrane transport protein encoded by the SLC7A5 gene. The large neutral amino acids transporter small subunit 2 or “LAT2” is a membrane transport protein encoded by the SLC7A8 gene.

In some embodiments, the Compounds of the Disclosure can be used to treat a “glutamine transporter-mediated disorder,” i.e., a disease, disorder, or condition wherein inhibition of ASCT2-, BOAT1-, SNAT1-, SNAT2-, SNAT3-, SNAT5-, SNAT7-, LAT1-, and/or LAT2-mediated glutamine transport provides a benefit. Such glutamine transporter-mediated disorders are represented by any pathological condition in which a glutamine transporter is known to play a role. In some embodiments, a glutamine transporter-mediated disorder is a proliferative disease such as cancer.

In some embodiments, the Compounds of the Disclosure can be used to treat an “ASCT2-mediated disorder,” i.e., a disease, disorder, or condition wherein inhibition of ASCT2-mediated amino acid, e.g., glutamine, transport provides a benefit. Such ASCT2-mediated disorders are represented by any pathological condition in which ASCT2 is known to play a role. In some embodiments, the ASCT2-mediated disorder is a proliferative disease such as cancer.

In some embodiments, the Compounds of the Disclosure can be used to treat a “BOAT1-mediated disorder,” i.e., a disease, disorder, or condition wherein inhibition of BOAT1-mediated amino acid, e.g., glutamine, transport provides a benefit. Such BOAT1-mediated disorders are represented by any pathological condition in which BOAT1 is known to play a role. In some embodiments, a BOAT1-mediated disorder is a proliferative disease such as cancer.

In some embodiments, the Compounds of the Disclosure can be used to treat a “SNAT1-mediated disorder,” i.e., a disease, disorder, or condition wherein inhibition of SNAT1-mediated amino acid, e.g., glutamine, transport provides a benefit. Such SNAT1-mediated disorders are represented by any pathological condition in which SNAT1 is known to play a role. In some embodiments, a SNAT1-mediated disorder is a proliferative disease such as cancer.

In some embodiments, the Compounds of the Disclosure can be used to treat a “SNAT2-mediated disorder,” i.e., a disease, disorder, or condition wherein inhibition of SNAT2-mediated amino acid, e.g., glutamine, transport provides a benefit. Such SNAT2-mediated disorders are represented by any pathological condition in which SNAT2 is known to play a role. In some embodiments, a SNAT2-mediated disorder is a proliferative disease such as cancer.

In some embodiments, the Compounds of the Disclosure can be used to treat a “SNAT3-mediated disorder,” i.e., a disease, disorder, or condition wherein inhibition of SNAT3-mediated amino acid, e.g., glutamine, transport provides a benefit. Such SNAT3-mediated disorders are represented by any pathological condition in which SNAT3 is known to play a role. In some embodiments, a SNAT3-mediated disorder is a proliferative disease such as cancer.

In some embodiments, the Compounds of the Disclosure can be used to treat a “SNAT5-mediated disorder,” i.e., a disease, disorder, or condition wherein inhibition of SNAT5-mediated amino acid, e.g., glutamine, transport provides a benefit. Such SNAT5-mediated disorders are represented by any pathological condition in which SNAT5 is known to play a role. In some embodiments, a SNAT5-mediated disorder is a proliferative disease such as cancer.

In some embodiments, the Compounds of the Disclosure can be used to treat a “SNAT7-mediated disorder,” i.e., a disease, disorder, or condition wherein inhibition of SNAT7-mediated amino acid, e.g., glutamine, transport provides a benefit. Such SNAT7-mediated disorders are represented by any pathological condition in which SNAT7 is known to play a role. In some embodiments, a SNAT7-mediated disorder is a proliferative disease such as cancer.

In some embodiments, the Compounds of the Disclosure can be used to treat a “LAT1-mediated disorder,” i.e., a disease, disorder, or condition wherein inhibition of LAT1-mediated amino acid, e.g., glutamine, transport provides a benefit. Such LAT1-mediated disorders are represented by any pathological condition in which LAT1 is known to play a role. In some embodiments, a LAT1-mediated disorder is a proliferative disease such as cancer.

In some embodiments, the Compounds of the Disclosure can be used to treat a “LAT2-mediated disorder,” i.e., a disease, disorder, or condition wherein inhibition of LAT2-mediated amino acid, e.g., glutamine, transport provides a benefit. Such LAT2-mediated disorders are represented by any pathological condition in which LAT2 is known to play a role. In some embodiments, a LAT2-mediated disorder is a proliferative disease such as cancer.

The term “biological sample” as used herein refers any tissue or fluid from a subject that is suitable for detecting the expression status and/or mutation status in a biomarker. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarkers using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practioner. In one embodiment of the disclosure, the biological sample comprises tumor cells or blood cells.

### VI. General Synthetic Methods

Compounds of the Disclosure can be prepared as shown in General Schemes 1 and 2. In General Scheme 1, a boronic acid having Formula A is made to react with a compound of Formula B in the presence of a palladium catalyst to give a compound of Formula I, wherein Q is Q-1 or Q-2.

In General Scheme 2, a benzyl bromide of Formula C is made to react with a compound of Formulae D, E, or F in the presence of a base, e.g., K₂CO₃, to give a compound of Formula I, wherein Q is Q-1, Q-2, or Q-3, respectively.

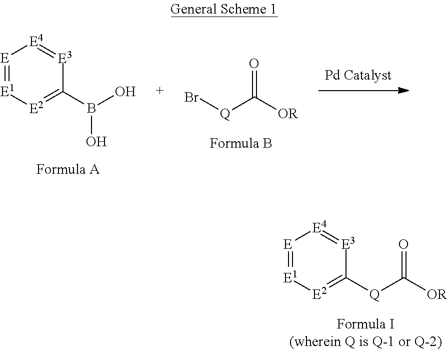

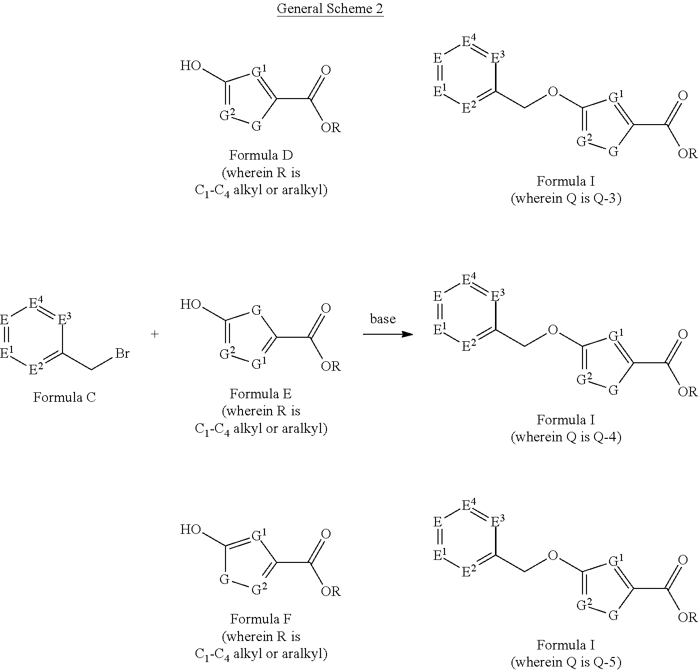

### EXAMPLES

### Example 1

### Synthesis of 3-([1,1′-Biphenyl]-4-ylmethoxy)isoxazole-5-carboxylic acid (Cpd. No. 47)

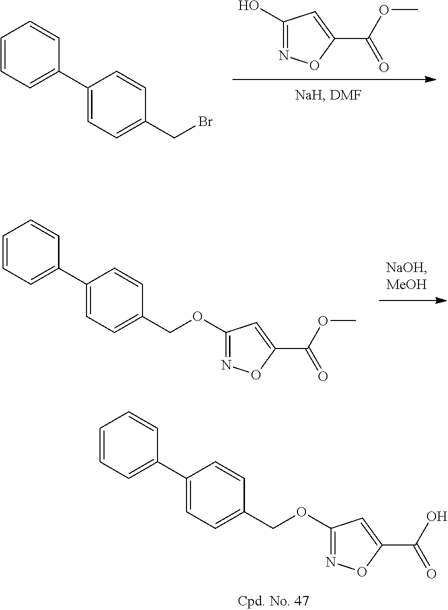

To a stirring solution of methyl 3-hydroxyisoxazole-5-carboxylate in DMF and NaH (1.1 eq.) was added 4-bromomethylmethylbiphenyl (1.1 eq.). The mixture was heated to 70 degrees C. and allowed to react overnight. The protected product was isolated using reverse-phase chromatography (Gilson), followed by base hydrolysis to give 3-([1,1′-Biphenyl]-4-ylmethoxy)isoxazole-5-carboxylic acid. The compound was characterized by LCMS (>99%), calculated mass=295; found m+Na=317.

### Example 2

### Synthesis of 3-([1,1′-Biphenyl]-4-ylmethoxy)-1H-pyrazole-5-carboxylic acid (Cpd. No. 45)

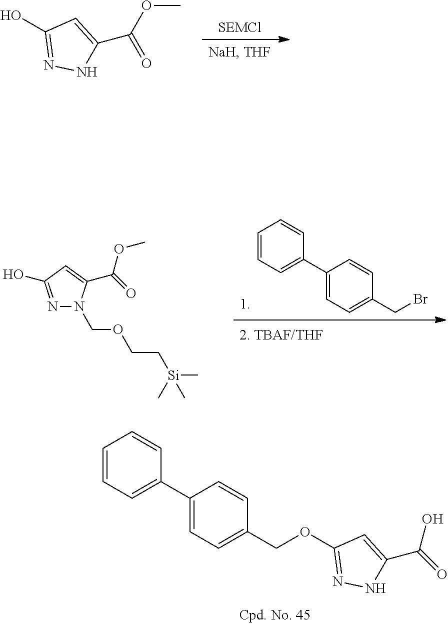

To a stirring solution of methyl 3-hydroxy-1H-pyrazole-5-carboxylate and NaH (1.1 eq.) in dry THF was added SEMCl. The protected pyrazole was isolated by flash chromatography on silica and reacted with 4-bromomethylmethylbiphenyl (1.1 eq.) with NaH in DMF. The protected biphenyl pyrazole was isolated by reverse-phase flash chromatography (Gilson), followed by deprotection of the SEM and methyl ester in a single step with TBAF in dry THF to give 3-([1,1′-biphenyl]-4-ylmethoxy)-1H-pyrazole-5-carboxylic acid. The compound was characterized by LCMS (>99%), calculated mass=294; found m+1=295.

### Example 3

### Synthesis of 5-([1,1′-Biphenyl]-4-yl)benzofuran-2-carboxylic acid (Cpd. No. 2)

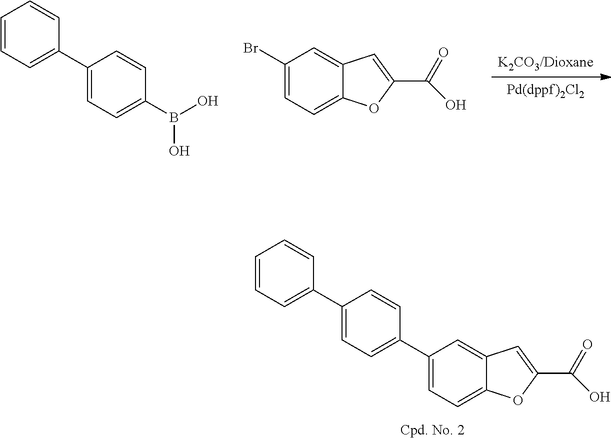 [SMILES: OB(O)c1ccc(-c2ccccc2)cc1 ; O=C(O)c1cc2cc(Br)ccc2o1 ; "K2CO3/Dioxane" ; "Pd(dppf)2Cl2" ; O=C(O)c1cc2cc(-c3ccc(-c4ccccc4)cc3)ccc2o1]

To a degassed solution of 5-bromobenzofuran-2-carboxylic acid, [1,1′-biphenyl]-4-ylboronic acid, potassium carbonate and dioxanes was added Pd catalyst. The reaction mixture was degassed for 5 minutes and heated to 80 degrees Celsius for 24 hrs. The reaction mixture was cooled and extracted from ethyl acetate, acidified in HCl, washed with brine, dried over sodium sulfate, and purified by reverse-phase chromatography (Gilson) to give 5-([1,1′-biphenyl]-4-yl)benzofuran-2-carboxylic acid. The compound was characterized by NMR and LCMS (>99%) calculated 314; found 314.

### Example 4

### Synthesis of 5-([1,1′-Biphenyl]-4-yl)-1H-pyrrolo[2,3-c]pyridine-2-carboxylic acid (Cpd. No. 10)

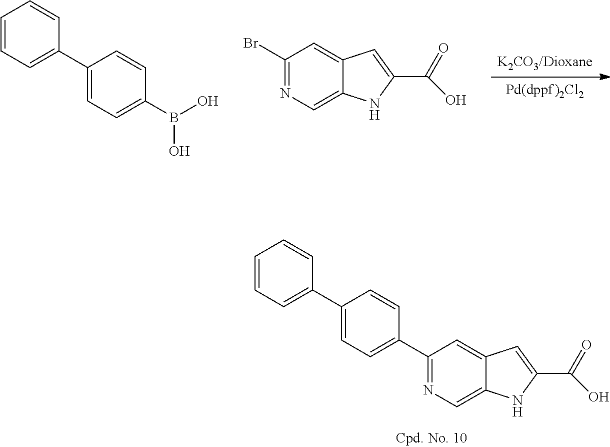 [SMILES: OB(O)c1ccc(-c2ccccc2)cc1 ; O=C(O)c1cc2cc(Br)ncc2[nH]1 ; "K2CO3/Dioxane" ; "Pd(dppf)2Cl2" ; O=C(O)c1cc2cc(-c3ccc(-c4ccccc4)cc3)ncc2[nH]1]

To a degassed solution of 5-bromo-1H-pyrrolo[2,3-c]pyridine-2-carboxylic acid, [1,1′-biphenyl]-4-ylboronic acid, potassium carbonate, and dioxanes was added Pd catalyst. The reaction mixture was degassed for 5 minutes and heated to 80 degrees Celsius for 72 hrs. The reaction mixture was cooled and extracted from aqueous ethyl acetate. The product was localized in the water layer. The water later was concentrated and reconstituted in DMSO. The crude product was purified by reverse-phase chromatography (Gilson) to give of 5-([1,1′-biphenyl]-4-yl)-1H-pyrrolo[2,3-c]pyridine-2-carboxylic acid. The compound was characterized by NMR and LCMS (>99%) calculated 314; found 314.

### Example 5

### Glutamine Uptake Inhibition Assay

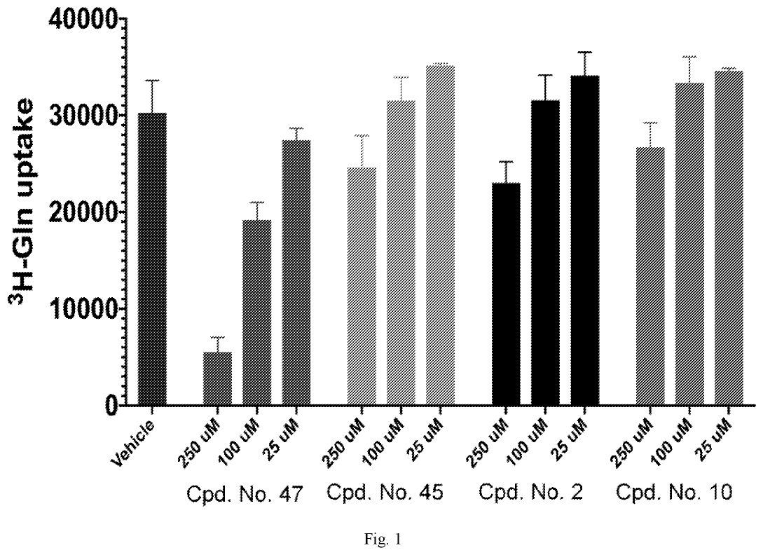

Twenty-four hours prior to assay, HEK293 cells were seeded at a density of 12K cells per well into a 96 well plate (coated with poly-D-Lysine). At the time of the assay (24 hrs later), these conditioned resulted in a confluence of approximately 50%. Accumulation of 3H-labeled glutamine (3H-GLN) in live cells was assessed by incubation for 15 minutes with vehicle or test reagent at the concentrations indicated. The final concentration of 3H-GLN was 500 nM. Each compound was evaluated in triplicate. For the assay, cells were rinsed with assay buffer (pH 6) three times (100 uL). Compounds were transferred in a single-add protocol and incubated for 15 minutes with 3H-GLN. After the uptake period, the supernatant was removed and cell monolayers were washed 3× with 100 μl assay buffer. Subsequently, cells were lysed with 50 μl 1M NaOH and 150 μl scintillation liquid was added to each well. The plate was incubated at room temperature for 20 minutes, transferred to 4° C. storage overnight, and read the next day using a top-count plate reader (Perkin Elmer). The results for representative Compounds of the Disclosure are provided in FIG. 1.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A compound of Formula I: 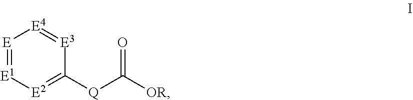
wherein:
R is selected from the group consisting of hydrogen, C₁-C₄ alkyl, and aralkyl;
E is selected from the group consisting of ═C(R⁵)—, ═C(R^{1a})—, and ═N—;
E¹ is selected from the group consisting of ═C(R⁵)—, ═C(R^{1a})—, and ═N—;
with the proviso that one of E or E¹ is ═C(R⁵)— and the other is ═C(R^{1a})— or ═N—;
E² is selected from the group consisting of ═C(R^{1b})— and ═N—;
E³ is selected from the group consisting of ═C(R^{1c})— and ═N—;
E⁴ is selected from the group consisting of ═C(R^{1d})— and ═N—;
R^{1a}, R^{1b}, R^{1c}, and R^{1d} are each independently selected from the group consisting of hydrogen, halogen, cyano, C₁-C₄ alkyl, C₁-C₄ haloalkyl, and C₁-C₄ alkoxy;
R⁵ is selected from the group consisting of optionally substituted aryl and optionally substituted heteroaryl;
Q is selected from the group consisting of: 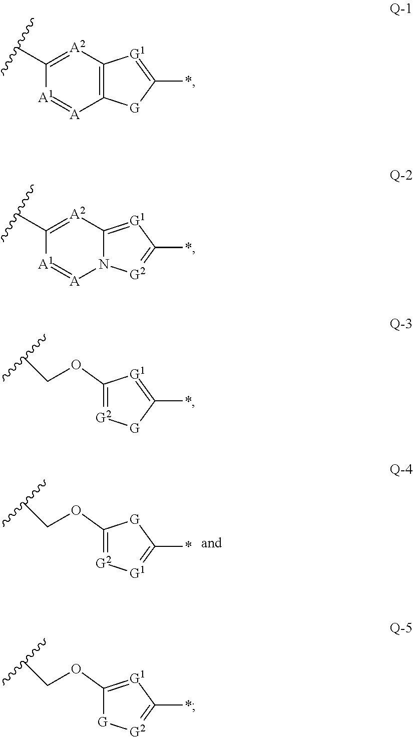
with the proviso that the bond designated with an “*” is attached to —C(═O)OR;
G is selected from the group consisting of —S—, —O—, and —N(R^{2a})—;
G¹ is selected from the group consisting of ═C(R^{3a})— and ═N—;
G² is selected from the group consisting of ═C(R^{3b})— and ═N—;
A is selected from the group consisting of ═C(R^{4a})— and ═N—;
A¹ is selected from the group consisting of ═C(R^{4b})— and ═N—;
A² is selected from the group consisting of ═C(R^{4c})— and ═N—;
R^{2a} is selected from the group consisting of hydrogen and C₁-C₄ alkyl;
R^{3a} and R^{3b} are independently selected from the group consisting of hydrogen, hydroxy, halo, C₁-C₄ alkyl, and C₁-C₄ haloalkyl; and
R^{4a}, R^{4b}, and R^{4c} are each independently selected from the group consisting of hydrogen, halogen, cyano, C₁-C₄ alkyl, C₁-C₄ haloalkyl, and C₁-C₄ alkoxy,
or a pharmaceutically acceptable salt or solvate thereof,
with the proviso that the compound is not:
5-([1,1′-biphenyl]-4-yl)-6-chloro-1H-pyrrolo[3,2-b]pyridine-2-carboxylic acid;
1-methyl-3-((4-(5-(trifluoromethyl)-1,2,4-oxadiazol-3-yl)benzyl)oxy)-1H-pyrazole-5-carboxylic acid; or
3-((4-(4-phenyl-1H-indol-1-yl)benzyl)oxy)isoxazole-5-carboxylic acid.

2. The compound of claim 1 of Formula II: 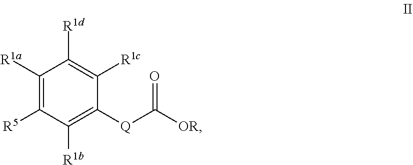
or a pharmaceutically acceptable salt or solvate thereof.

3. The compound of claim 1 of Formula III: 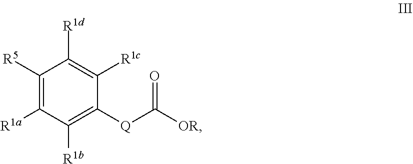
or a pharmaceutically acceptable salt or solvate thereof.

4. The compound of claim 1, wherein R⁵ is optionally substituted heteroaryl, or a pharmaceutically acceptable salt or solvate thereof.

5. The compound of claim 1, wherein R⁵ is optionally substituted aryl, or a pharmaceutically acceptable salt or solvate thereof.

6. The compound of claim 5 of Formula IV: 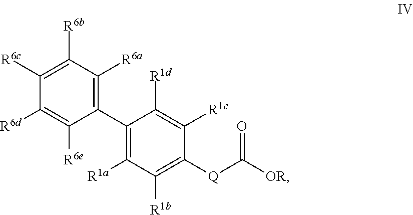
wherein R^{6a}, R^{6b}, R^{6c}, R^{6d}, and R^{6e} are each independently selected from the group consisting of hydrogen, halogen, cyano, C₁-C₄ alkyl, C₁-C₄ haloalkyl, and C₁-C₄ alkoxy, or a pharmaceutically acceptable salt or solvate thereof.

7. The compound of claim 6, wherein R^{1a}, R^{1b}, R^{1c}, R^{1d}, R^{6d}, and R^{6e} are each hydrogen, or a pharmaceutically acceptable salt or solvate thereof.

8. The compound of claim 1, wherein Q is Q-1, or a pharmaceutically acceptable salt or solvate thereof.

9. The compound of claim 7, wherein Q-1 is selected from the group consisting of: 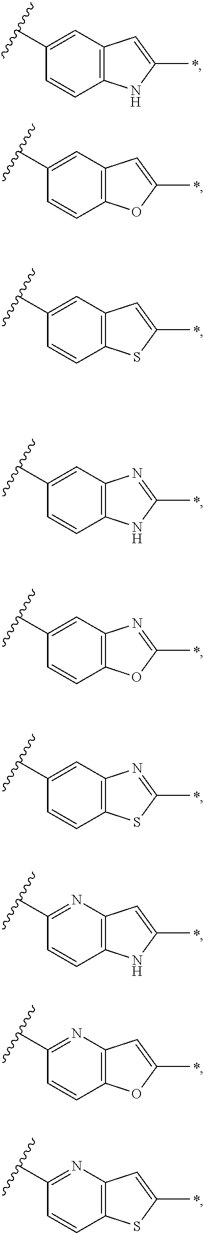 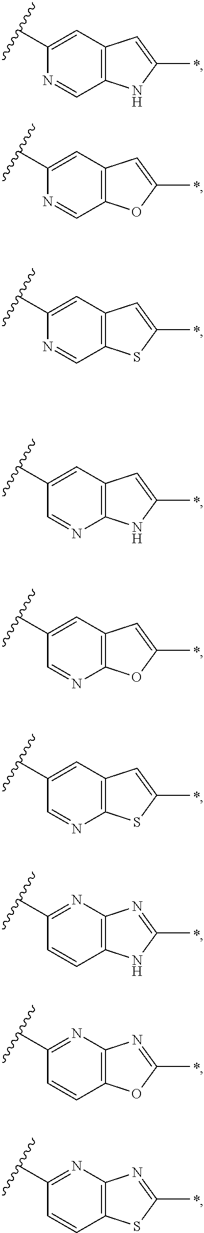 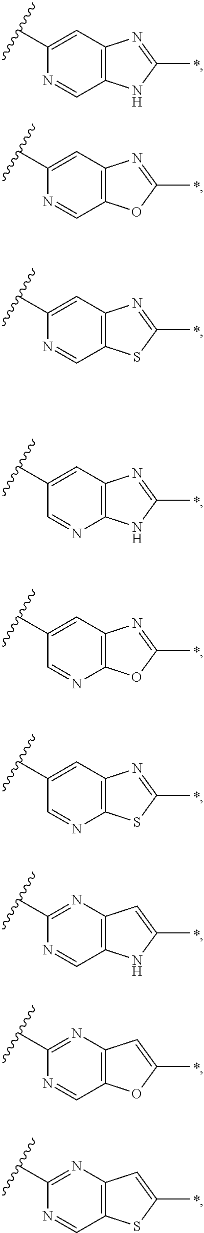 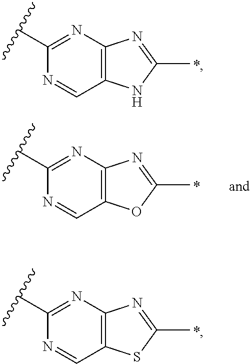 [SMILES: *c1ncc2[nH]c(C)nc2n1 ; *c1ncc2oc(C)nc2n1 ; *c1ncc2sc(C)nc2n1]
or a pharmaceutically acceptable salt or solvate thereof.

10. The compound of claim 1, wherein Q is Q-2, or a pharmaceutically acceptable salt or solvate thereof.

11. The compound of claim 10, wherein Q-2 is selected from the group consisting of 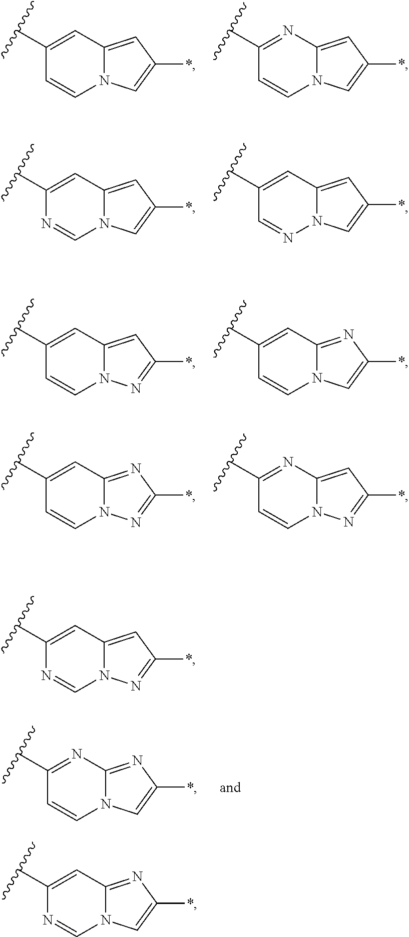
or a pharmaceutically acceptable salt or solvate thereof.

12. The compound of claim 1, wherein Q is Q-3, or a pharmaceutically acceptable salt or solvate thereof.

13. The compound of claim 12, wherein Q-3 is selected from the group consisting of: 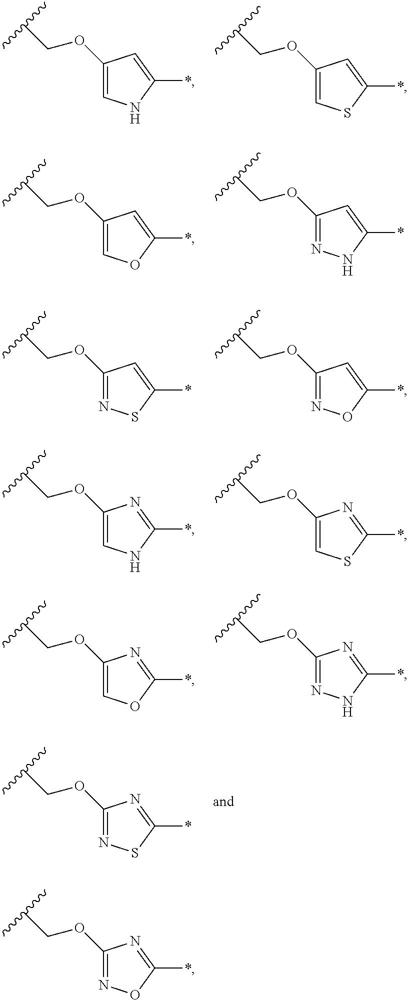
or a pharmaceutically acceptable salt or solvate thereof.

14. The compound of claim 1, wherein Q is Q-4, or a pharmaceutically acceptable salt or solvate thereof.

15. The compound of claim 14, wherein Q-4 is selected from the group consisting of: 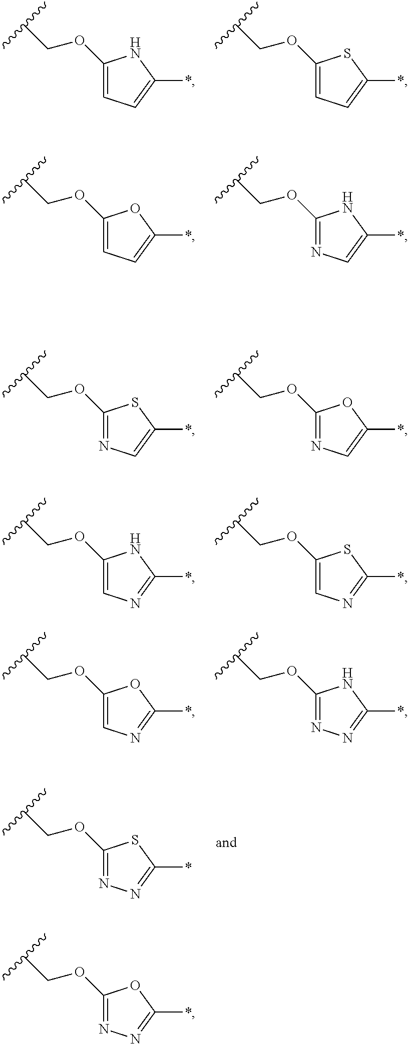
or a pharmaceutically acceptable salt or solvate thereof.

16. The compound of claim 1, wherein Q is Q-5, or a pharmaceutically acceptable salt or solvate thereof.

17. The compound of claim 16, wherein Q-5 is selected from the group consisting of: 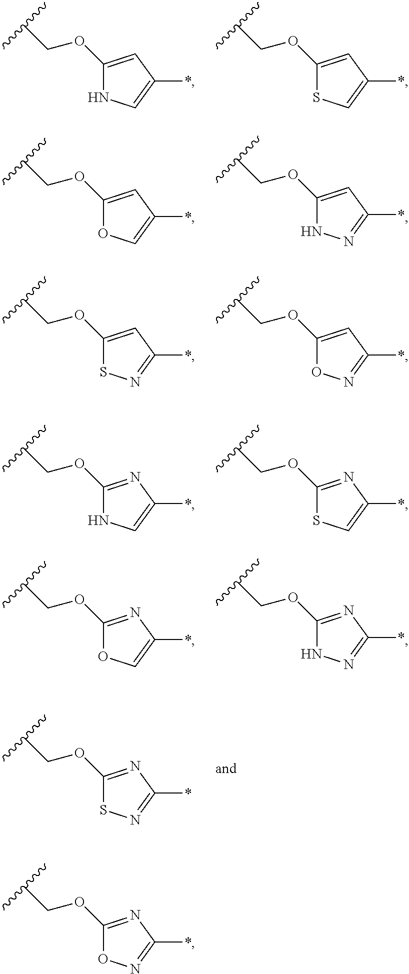
or a pharmaceutically acceptable salt or solvate thereof.

18. The compound of claim 1, wherein R is hydrogen, or a pharmaceutically acceptable salt or solvate thereof.

19. The compound of claim 1 selected from the group consisting of:
5-([1,1′-biphenyl]-4-yl)-1H-indole-2-carboxylic acid;
5-([1,1′-biphenyl]-4-yl)benzofuran-2-carboxylic acid;
5-([1,1′-biphenyl]-4-yl)benzo[b]thiophene-2-carboxylic acid;
5-([1,1′-biphenyl]-4-yl)-1H-benzo[d]imidazole-2-carboxylic acid;
5-([1,1′-biphenyl]-4-yl)benzo[d]oxazole-2-carboxylic acid;
5-([1,1′-biphenyl]-4-yl)benzo[d]thiazole-2-carboxylic acid;
5-([1,1′-biphenyl]-4-yl)-1H-pyrrolo[3,2-b]pyridine-2-carboxylic acid;
5-([1,1′-biphenyl]-4-yl)furo[3,2-b]pyridine-2-carboxylic acid;
5-([1,1′-biphenyl]-4-yl)thieno[3,2-b]pyridine-2-carboxylic acid;
5-([1,1′-biphenyl]-4-yl)-1H-pyrrolo[2,3-c]pyridine-2-carboxylic acid;
5-([1,1′-biphenyl]-4-yl)furo[2,3-c]pyridine-2-carboxylic acid;
5-([1,1′-biphenyl]-4-yl)thieno[2,3-c]pyridine-2-carboxylic acid;
5-([1,1′-biphenyl]-4-yl)-1H-pyrrolo[2,3-b]pyridine-2-carboxylic acid;
5-([1,1′-biphenyl]-4-yl)furo[2,3-b]pyridine-2-carboxylic acid;
5-([1,1′-biphenyl]-4-yl)thieno[2,3-b]pyridine-2-carboxylic acid;
5-([1,1′-biphenyl]-4-yl)-1H-imidazo[4,5-b]pyridine-2-carboxylic acid;
5-([1,1′-biphenyl]-4-yl)oxazolo[4,5-b]pyridine-2-carboxylic acid;
5-([1,1′-biphenyl]-4-yl)thiazolo[4,5-b]pyridine-2-carboxylic acid;
6-([1,1′-biphenyl]-4-yl)-3H-imidazo[4,5-c]pyridine-2-carboxylic acid;
6-([1,1′-biphenyl]-4-yl)oxazolo[5,4-c]pyridine-2-carboxylic acid;
6-([1,1′-biphenyl]-4-yl)thiazolo[5,4-c]pyridine-2-carboxylic acid;
6-([1,1′-biphenyl]-4-yl)-3H-imidazo[4,5-b]pyridine-2-carboxylic acid;
6-([1,1′-biphenyl]-4-yl)oxazolo[5,4-b]pyridine-2-carboxylic acid;
6-([1,1′-biphenyl]-4-yl)thiazolo[5,4-b]pyridine-2-carboxylic acid;
2-([1,1′-biphenyl]-4-yl)-5H-pyrrolo[3,2-d]pyrimidine-6-carboxylic acid;
2-([1,1′-biphenyl]-4-yl)furo[3,2-d]pyrimidine-6-carboxylic acid;
2-([1,1′-biphenyl]-4-yl)thieno[3,2-d]pyrimidine-6-carboxylic acid;
2-([1,1′-biphenyl]-4-yl)-7H-purine-8-carboxylic acid;
5-([1,1′-biphenyl]-4-yl)oxazolo[4,5-d]pyrimidine-2-carboxylic acid;
5-([1,1′-biphenyl]-4-yl)thiazolo[4,5-d]pyrimidine-2-carboxylic acid;
7-([1,1′-biphenyl]-4-yl)indolizine-2-carboxylic acid;
2-([1,1′-biphenyl]-4-yl)pyrrolo[1,2-a]pyrimidine-7-carboxylic acid;
3-([1,1′-biphenyl]-4-yl)pyrrolo[1,2-c]pyrimidine-6-carboxylic acid;
3-([1,1′-biphenyl]-4-yl)pyrrolo[1,2-b]pyridazine-6-carboxylic acid;
5-([1,1′-biphenyl]-4-yl)pyrazolo[1,5-a]pyridine-2-carboxylic acid;
7-([1,1′-biphenyl]-4-yl)imidazo[1,2-a]pyridine-2-carboxylic acid;
7-([1,1′-biphenyl]-4-yl)-[1,2,4]triazolo[1,5-a]pyridine-2-carboxylic acid;
5-([1,1′-biphenyl]-4-yl)pyrazolo[1,5-a]pyrimidine-2-carboxylic acid;
5-([1,1′-biphenyl]-4-yl)pyrazolo[1,5-c]pyrimidine-2-carboxylic acid;
7-([1,1′-biphenyl]-4-yl)imidazo[1,2-a]pyrimidine-2-carboxylic acid;
7-([1,1′-biphenyl]-4-yl)imidazo[1,2-c]pyrimidine-2-carboxylic acid;
4-([1,1′-biphenyl]-4-ylmethoxy)-1H-pyrrole-2-carboxylic acid;
4-([1,1′-biphenyl]-4-ylmethoxy)thiophene-2-carboxylic acid;
4-([1,1′-biphenyl]-4-ylmethoxy)furan-2-carboxylic acid;
3-([1,1′-biphenyl]-4-ylmethoxy)-1H-pyrazole-5-carboxylic acid;
3-([1,1′-biphenyl]-4-ylmethoxy)isothiazole-5-carboxylic acid;
3-([1,1′-biphenyl]-4-ylmethoxy)isoxazole-5-carboxylic acid;
4-([1,1′-biphenyl]-4-ylmethoxy)-1H-imidazole-2-carboxylic acid;
4-([1,1′-biphenyl]-4-ylmethoxy)thiazole-2-carboxylic acid;
4-([1,1′-biphenyl]-4-ylmethoxy)oxazole-2-carboxylic acid;
3-([1,1′-biphenyl]-4-ylmethoxy)-1H-1,2,4-triazole-5-carboxylic acid;
3-([1,1′-biphenyl]-4-ylmethoxy)-1,2,4-thiadiazole-5-carboxylic acid;
3-([1,1′-biphenyl]-4-ylmethoxy)-1,2,4-oxadiazole-5-carboxylic acid;
5-([1,1′-biphenyl]-4-ylmethoxy)-1H-pyrrole-2-carboxylic acid;
5-([1,1′-biphenyl]-4-ylmethoxy)thiophene-2-carboxylic acid;
5-([1,1′-biphenyl]-4-ylmethoxy)furan-2-carboxylic acid;
2-([1,1′-biphenyl]-4-ylmethoxy)-1H-imidazole-5-carboxylic acid;
2-([1,1′-biphenyl]-4-ylmethoxy)thiazole-5-carboxylic acid;
2-([1,1′-biphenyl]-4-ylmethoxy)oxazole-5-carboxylic acid;
5-([1,1′-biphenyl]-4-ylmethoxy)-1H-imidazole-2-carboxylic acid;
5-([1,1′-biphenyl]-4-ylmethoxy)thiazole-2-carboxylic acid;
5-([1,1′-biphenyl]-4-ylmethoxy)oxazole-2-carboxylic acid;
5-([1,1′-biphenyl]-4-ylmethoxy)-4H-1,2,4-triazole-3-carboxylic acid;
5-([1,1′-biphenyl]-4-ylmethoxy)-1,3,4-thiadiazole-2-carboxylic acid;
5-([1,1′-biphenyl]-4-ylmethoxy)-1,3,4-oxadiazole-2-carboxylic acid;
5-([1,1′-biphenyl]-4-ylmethoxy)-1H-pyrrole-3-carboxylic acid;
5-([1,1′-biphenyl]-4-ylmethoxy)thiophene-3-carboxylic acid;
5-([1,1′-biphenyl]-4-ylmethoxy)furan-3-carboxylic acid;
5-([1,1′-biphenyl]-4-ylmethoxy)-1H-pyrazole-3-carboxylic acid;
5-([1,1′-biphenyl]-4-ylmethoxy)isothiazole-3-carboxylic acid;
5-([1,1′-biphenyl]-4-ylmethoxy)isoxazole-3-carboxylic acid;
2-([1,1′-biphenyl]-4-ylmethoxy)-1H-imidazole-4-carboxylic acid;
2-([1,1′-biphenyl]-4-ylmethoxy)thiazole-4-carboxylic acid;
2-([1,1′-biphenyl]-4-ylmethoxy)oxazole-4-carboxylic acid;
5-([1,1′-biphenyl]-4-ylmethoxy)-1H-1,2,4-triazole-3-carboxylic acid;
5-([1,1′-biphenyl]-4-ylmethoxy)-1,2,4-thiadiazole-3-carboxylic acid; and
5-([1,1′-biphenyl]-4-ylmethoxy)-1,2,4-oxadiazole-3-carboxylic acid,
or a pharmaceutically acceptable salt or solvate thereof.

20. A pharmaceutical composition comprising the compound of claim 1 or a pharmaceutically acceptable salt or solvate thereof, and a pharmaceutically acceptable carrier.

21. A method of treating cancer a subject in need thereof, the method comprising administering to the subject a therapeutically effective amount of the compound of claim 1, or a pharmaceutically acceptable salt or solvate thereof.

22. The method of claim 21, wherein the cancer is a solid tumor.

23. The method of claim 21, wherein the cancer is a hematological cancer.

24. The method of claim 21, wherein the cancer is any one or more of the cancers of Table 3.

25. The method of claim 21, wherein the cancer is any one or more of the cancers of Table 4.

26. The method of claim 21, further comprising administering to the subject a therapeutically effective amount of one or more optional therapeutic agents useful in the treatment of cancer.

27-43. (canceled)

44. A therapeutic or prophylactic agent for cancer, which comprises the compound of claim 1, or a pharmaceutically acceptable salt thereof.

45-50. (canceled)

51. A method of treating a subject having cancer, the method comprising administering a therapeutically effective amount of a compound of claim 1, or a pharmaceutically acceptable salt thereof, to the subject if a mutation in BRAF, KRAS, p53, or PI3KCA, or a combination thereof, is present in a biological sample of the subject.

52. A method of identifying whether a subject having cancer as a candidate for treatment with a compound of claim 1, or a pharmaceutically acceptable salt or solvate thereof, the method comprising:
(a) identifying the subject as being a candidate for treatment if a mutation in BRAF, KRAS, p53, or PI3KCA, or a combination thereof is present in a biological sample of the subject; or
(b) identifying the subject as not being a candidate for treatment if a mutation in BRAF, KRAS, p53, or PI3KCA, or a combination thereof is absent in a biological sample of the subject.

53. A method of predicting treatment outcome in a subject having cancer, the method comprising:
(a) if a mutation in BRAF, KRAS, p53, or PI3KCA, or a combination thereof is present in a biological sample of the subject, then administering a compound of claim 1, or a pharmaceutically acceptable salt thereof, to the subject will likely cause a favorable therapeutic response; and
(b) if a mutation in BRAF, KRAS, p53, or PI3KCA, or a combination thereof is absent in the biological sample, then administering a compound of any one of claims 1-19, or a pharmaceutically acceptable salt thereof, to the subject will likely cause an unfavorable therapeutic response.

54-56. (canceled)
